SEQUENCE LISTING (14 sequences)

SEQ ID NO: 1 - protein, 859 aa, Homo sapiens
MACLHETRTPSPSFGGFVSTLSEASMRKLDPDTSDCTPEKDLTPTHVLQLHEQDAGGPGGAAGSPESRASRVRADEVRLQCQSGSGFLEGLFGCLRPVWTMIGKAYSTEHKQQQEDLWEVPFEEILDLQWVGSGAQGAVFLGRFHGEEVAVKKVRDLKETDIKHLRKLKHPNIITFKGVCTQAPCYCILMEFCAQGQLYEVLRAGRPVTPSLLVDWSMGIAGGMNYLHLHKIIHRDLKSPNMLITYDDVVKISDFGTSKELSDKSTKMSFAGTVAWMAPEVIRNEPVSEKVDIWSFGVVLWELLTGEIPYKDVDSSAIIWGVGSNSLHLPVPSSCPDGFKILLRQCWNSKPRNRPSFRQILLHLDIASADVLSTPQETYFKSQAEWREEVKLHFEKIKSEGTCLHRLEEELVMRRREELRHALDIREHYERKLERANNLYMELNALMLQLELKERELLRREQALERRCPGLLKPHPSRGLLHGNTMEKLIKKRNVPQKLSPHSKRPDILKTESLLPKLDAALSGVGLPGCPKGPPSPGRSRRGKTRHRKASAKGSCGDLPGLRTAVPPHEPGGPGSPGGLGGGPSAWEACPPALRGLHHDLLLRKMSSSSPDLLSAALGSRGRGATGGAGDPGSPPPARGDTPPSEGSAPGSTSPDSPGGAKGEPPPPVGPGEGVGLLGTGREGTSGRGGSRAGSQHLTPAALLYRAAVTRSQKRGISSEEEEGEVDSEVELTSSQRWPQSLNMRQSLSTFSSENPSDGEEGTASEPSPSGTPEVGSTNTDERPDERSDDMCSQGSEIPLDPPPSEVIPGPEPSSLPIPHQELLRERGPPNSEDSDCDSTELDNSNSVDALRPPASLPP

SEQ ID NO: 2 - protein, 888 aa, Mus musculus
MACLHETRTPSPSFGGFVSTLSEASMRKLDPDTSDCTPEKDLTPTQCVLRDVVPLGGQGGGGPSPSPGGEPPPEPFANSVLQLHEQDTGGPGGATGSPESRASRVRADEVRLQCQSGSGFLEGLFGCLRPVWTMIGKAYSTEHKQQQEDLWEVPFEEILDLQWVGSGAQGAVFLGRFHGEEVAVKKVRDLKETDIKHLRKLKHPNIITFKGVCTQAPCYCILMEFCAQGQLYEVLRAGRPVTPSLLVDWSMGIAGGMNYLHLHKIIHRDLKSPNMLITYDDVVKISDFGTSKELSDKSTKMSFAGTVAWMAPEVIRNEPVSEKVDIWSFGVVLWELLTGEIPYKDVDSSAIIWGVGSNSLHLPVPSSCPDGFKILLRQCWNSKPRNRPSFRQILLHLDIASADVLSTPQETYFKSQAEWREEVKLHFEKIKSEGTCLHRLEEELVMRRREELRHALDIREHYERKLERANNLYMELNALMLQLELKERELLRREQALERRCPGLLKSHPSRGLLHGNTMEKLIKKRNVPQKLSPHSKRPDILKTESLLPKLDAALSGVGLPGCPKGPPSPGRSRRGKTRHRKASAKGSCGDLPGLRAALPPHEPGGLGSPGGLGVGPSAWDACPPALRGLHHDLLLRKMSSSSPDLLSAALGARGRGATGGARDPGSPPPPQGDTPPSEGSAPGSTSPDSPGGAKGEPPPPVGPGEGVGLLGTGREGTAGRGGNRAGSQHLTPAALLYRAAVTRSQKRGISSEEEEGEVDSEVELPPSQRWPQGPNMRQSLSTFSSENPSDVEEGTASEPSPSGTPEVGSTNTDERPDERSDDMCSQGSEIPLDLPTSEVVPEREASSLPMQHQDGQGPNPEDSDCDSTELDNSNSIDALRPPASLPP

SEQ ID NO: 3 - DNA, 21 bp, Artificial Sequence
  Description of Artificial Sequence Synthetic oligonucleotide
ttggatgaagccattagacta

SEQ ID NO: 4 - DNA, 21 bp, Artificial Sequence
  Description of Artificial Sequence Synthetic oligonucleotide
acctttaatggacaacattaa

SEQ ID NO: 5 - DNA, 21 bp, Artificial Sequence
  Description of Artificial Sequence Synthetic oligonucleotide
aaggattagctttgtatcata

SEQ ID NO: 6 - DNA, 21 bp, Artificial Sequence
  Description of Artificial Sequence Synthetic oligonucleotide
cccgcatgtgtctgtattcaa

SEQ ID NO: 7 - DNA, 23 bp, Artificial Sequence
  Description of Artificial Sequence Synthetic primer
ccagtaacattgtagtcaagtct

SEQ ID NO: 8 - DNA, 18 bp, Artificial Sequence
  Description of Artificial Sequence Synthetic primer
tggtcttccgcttggtat

SEQ ID NO: 9 - DNA, 18 bp, Artificial Sequence
  Description of Artificial Sequence Synthetic primer
cgccttctatcgccttct

SEQ ID NO: 10 - DNA, 22 bp, Artificial Sequence
  Description of Artificial Sequence Synthetic oligonucleotide
acatcgtagtcaagtctgattt

SEQ ID NO: 11 - DNA, 22 bp, Artificial Sequence
  Description of Artificial Sequence Synthetic oligonucleotide
atcagacttgactacgatgttt

SEQ ID NO: 12 - protein, 15 aa, Homo sapiens
  MOD_RES (6)..(6): Phosphorylated Thr
PEKDLTPTHVLQLHC

SEQ ID NO: 13 - protein, 15 aa, Unknown
  Description of Unknown Homo sapiens or Mus sp. peptide
RNVPQKLSPHSKRPC

SEQ ID NO: 14 - protein, 15 aa, Unknown
  Description of Unknown Homo sapiens or Mus sp. peptide
HRDLKSPNMLITYDC

(19) United States
(12) Patent Application Publication
(10) Pub. No.: US 2015/0361184 A1
(43) Pub. Date: Dec. 17, 2015

(54) METHODS OF MODULATING DLK STABILITY

(71) Applicant: GENENTECH, INC., South San Francisco, CA (US)

(72) Inventors: Daisy BUSTOS, South San Francisco, CA (US); Sarah HUNTWORK-RODRIGUEZ, South San Francisco, CA (US); Donald KIRKPATRICK, South San Francisco, CA (US); Joseph Wesley LEWCOCK, South San Francisco, CA (US); Arundhati Sengupta GHOSH, South San Francisco, CA (US)

(73) Assignee: GENENTECH, INC., South San Francisco, CA (US)

(21) Appl. No.: 14/839,813

(22) Filed: Aug. 28, 2015

Related U.S. Application Data
(63) Continuation of application No. PCT/US2014/019122, filed on Feb. 27, 2014.
(60) Provisional application No. 61/770,959, filed on Feb. 28, 2013.

(51) Int. Cl. C07K 16/40; C12N 15/113; A61K 31/7052; A61K 45/06; A61K 39/395; G01N 33/573; A61K 38/02

(52) U.S. Cl. CPC C07K 16/40; G01N 33/573; C12N 15/1137; A61K 38/02; A61K 45/06; A61K 39/3955; A61K 31/7052; G01N 2333/912; G01N 2440/14; G01N 2800/7004; C07K 2317/55; C07K 2317/54; C07K 2317/76; C12N 2310/14

(57) ABSTRACT

The invention provides for methods of decreasing dual leucine zipper kinase (DLK) stability in a neuron, or decreasing or inhibiting the phosphorylation of certain amino acid residues of DLK, comprising administering to a neuron, or portion thereof, an agent which decreases or inhibits the phosphorylation of DLK and decreases the stability of DLK as well as methods for inhibiting or preventing neuronal degeneration in a patient by administering to a patient an agent which inhibits phosphorylation of dual leucine zipper kinase (DLK).

### RELATED APPLICATIONS

This application claims the benefit of U.S. Provisional Application No. 61/770,959, filed on 28 Feb. 2013, which is incorporated by reference herein in its entirety.

### FIELD OF THE INVENTION

The present invention relates to methods of preventing neuronal degeneration by decreasing the stability of the dual leucine zipper kinase (DLK) via inhibition of DLK phosphorylation.

### BACKGROUND

Axon degeneration and neuronal cell death occur during development to refine neuronal connections (Oppenheim, R. W. Annual review of neuroscience 14, 453-501 (1991); Luo, L. & O'Leary, D. D. Annual review of neuroscience 28, 127-156 (2005)), after injury to clear damaged cells (Quigley, H. A. et al. Investigative ophthalmology & visual science 36, 774-786 (1995)), and in neurodegenerative diseases such as Parkinson's Disease, Amyotrophic Lateral Sclerosis (ALS), and Alzheimer's Disease (Vila, M. & Przedborski, S. Nature reviews. Neuroscience 4, 365-375 (2003)). While the factors that trigger neurodegeneration in these settings vary widely, conserved signaling events that appear to be common to many neurodegenerative contexts have been identified that initiate axon degeneration and neuronal apoptosis. One example of this is the Jun N-terminal kinases (JNKs), which act upstream of Bax and c-Jun in activating axon degeneration and neuronal apoptosis in both development (Ham, J. et al. Neuron 14, 927-939 (1995); Kuan, C. Y. et al. Neuron 22, 667-676 (1999); Southwell, D. G. et al. Nature 491, 109-113 (2012); White, F. A., Keller-Peck, C. R., Knudson, C. M., Korsmeyer, S. J. & Snider, W. D. The Journal of neuroscience: the official journal of the Society for Neuroscience 18, 1428-1439 (1998)) and as part of neurodegenerative disease pathology (Hunot, S. et al. PNAS 101, 665-670 (2004); Martin, L. J. Journal of neuropathology and experimental neurology 58, 459-471 (1999); Vila, M. et al. PNAS 98, 2837-2842 (2001); Yao, M., Nguyen, T. V. & Pike, C. J. The Journal of neuroscience: the official journal of the Society for Neuroscience 25, 1149-1158 (2005)). In each of these settings, Bax-dependent caspase activation appears necessary to carry out programmed cell death and axon degeneration downstream of JNK activation (Simon, D. J. et al. The Journal of neuroscience: the official journal of the Society for Neuroscience 32, 17540-17553 (2012); Pettmann, B. & Henderson, C. E. Neuron 20, 633-647 (1998); Gagliardini, V. et al. Science 263, 826-828 (1994); Yuan, J. & Yankner, B. A. Nature 407, 802-809 (2000)).

Dual leucine zipper bearing kinase (DLK) is an evolutionarily conserved, highly neuron-specific member of the mixed lineage kinase (MLK) family that is required for stress-induced neuronal JNK activation (Hirai, S. et al. Gene expression patterns: GEP 5, 517-523 (2005); Ghosh, A. S. et al. The Journal of cell biology 194, 751-764 (2011); Watkins, T. A. et al. DLK initiates a transcriptional program that couples apoptotic and regenerative responses to axonal injury. In Press (2013); Chen, X. et al. The Journal of neuroscience: the official journal of the Society for Neuroscience 28, 672-680 (2008). Loss of DLK in mammals is sufficient to attenuate apoptosis and axon degeneration in development and following axon injury (Ghosh, A. S. et al. (2011); Watkins, T. A. et al. In Press (2013); Chen, X. et al. (2008); Miller, B. R. et al. Nature neuroscience 12, 387-389 (2009)). In invertebrates, a distinct function for DLK was identified through successive genetic screens that demonstrated that the PHR family of E3 ubiquitin ligases (PAM/highwire/RPM-1) negatively regulates DLK levels to control synapse development (Collins, C. A., Wairkar, Y. P., Johnson, S. L. & DiAntonio, A. Neuron 51, 57-69 (2006); Nakata, K. et al. Cell 120, 407-420 (2005)). Overexpression of the deubiquitinating enzyme (DUB) fat facets (faf) yields a synapse phenotype similar to highwire mutants, suggesting that Faf may counteract PHR ligases to positively regulate DLK levels (Collins, C. A., Wairkar, Y. P., Johnson, S. L. & DiAntonio, A. (2006)). A similar mechanism appears to regulate DLK following nerve injury in Drosophila, where DLK levels are rapidly up-regulated in a PHR-dependent fashion (Xiong, X. et al. The Journal of cell biology 191, 211-223 (2010)). In C. elegans, DLK activity following injury is also regulated via heterodimerization with a shorter DLK isoform that restricts DLK activation to damaged regions of the neuron (Yan, D. & Jin, Y. Neuron 76, 534-548 (2012)).

Despite the mechanistic knowledge gained through studies in invertebrate systems, little is known about whether DLK is similarly regulated in mammalian neurons. Mice lacking expression of Phr1, the mouse PHR ubiquitin ligase, show no gross change in DLK abundance in whole brain, and loss of DLK fails to suppress the axon pathfinding defects observed in Phr1 mutants (Bloom, A. J., Miller, B. R., Sanes, J. R. & DiAntonio, A. Genes & development 21, 2593-2606 (2007)). However, regulation of DLK levels by the ubiquitin-proteasome system in a mammalian neuronal injury paradigm has not been rigorously examined.

DLK is a MAP3K that senses neuronal damage and triggers both degenerative and regenerative signaling (Ghosh, A. S. et al. (2011); Watkins, T. A. et al. In Press (2013); Miller, B. R. et al. (2009); Shin, J. E. et al. Neuron 74, 1015-1022 (2012)). Loss of DLK is sufficient to completely suppress JNK activation and downstream responses in a strikingly wide variety of neuronal stress paradigms (Watkins, T. A. et al. In Press (2013), but it has been unclear how DLK is itself regulated by neuronal stress in mammalian neurons. The Examples described herein demonstrate that in mammalian neurons: (1) DLK quantity rapidly increases early in the response to neuronal stress, (2), DLK levels are controlled by a positive feedback loop in which JNK activity regulates phosphorylation of a number of sites within DLK that modulate protein stability (3) DLK levels are regulated by Phr1 and USP9X, (4) alteration in DLK protein stability can occur independent of stress-induced activation of DLK signaling, and (5) DLK protein quantity directly controls the amount of downstream signaling.

### SUMMARY

The invention provides for methods of modulating DLK stability via inhibition of phosphorylation of dual leucine zipper kinase (DLK). The invention also provides for methods of inhibiting or preventing neuronal degeneration in a patient via administration of an agent which inhibits the phosphorylation of DLK and in particular, inhibits the phosphorylation of specific amino acid residues of DLK.

The invention is based on the observation that DLK levels reproducibly increase following various types of neuronal stress. Not to be bound by theory, Phr1 and the de-ubiquitinase (DUB) USP9X function to tightly regulate the abundance of DLK protein in neurons, though neither regulates DLK activity. Following neuronal-injury-dependent activation, DLK becomes hyper-phosphorylated, which results in increased protein stability via specific JNK dependent phosphorylation events outside the kinase domain that are distinct from those which regulate DLK kinase activity. Thus, DLK pathway activation generates a feedback mechanism that increases the levels of DLK protein, which in turn enhances phosphorylation of downstream targets and converts graded or local DLK signaling into a more complete response that allows neurons to properly react to injury.

In one embodiment, the invention provides a method for decreasing dual leucine zipper kinase (DLK) stability in a neuron, the method comprising administering to a neuron, or portion thereof, an agent which decreases or inhibits the phosphorylation of DLK and decreases the stability of DLK. In certain embodiments, the agent inhibits or decreases the phosphorylation of a specific DLK amino acid residue or residues.

In other embodiments, the invention provides a method for inhibiting or decreasing the phosphorylation of certain amino acid residues of dual leucine zipper kinase (DLK), the method comprising administering to a neuron or portion thereof an agent which inhibits or decreases the phosphorylation of certain amino acid residues of DLK, wherein the inhibition or decrease of phosphorylation results in a decrease of DLK protein stability.

In other embodiments, the invention provides a method for inhibiting or preventing neuronal degeneration in a patient wherein the method comprises administering to a patient an agent which inhibits or decreases phosphorylation of dual leucine zipper kinase (DLK), wherein the inhibition or decrease of phosphorylation decreases the stability of DLK. In certain embodiments, the agent inhibits or decreases the phosphorylation of a specific DLK amino acid residue or residues.

In other embodiments, the invention provides for a method for detecting stress dependent or pro-apopototic DLK activity in a neuron, the method comprising: (a) contacting a biological sample with an antibody which specifically recognizes a phosphorylated form of DLK; and (b) detecting binding of the antibody to the phosphorylated form of DLK within the biological sample, wherein binding by the antibody indicates stress dependent or pro-apoptotic DLK activity. In certain embodiments, the method further comprises measuring the binding of the antibody to the phosphorylated form of DLK, wherein an increase in binding of the antibody in the biological sample relative to a control is indicative of stress dependent or pro-apoptotic DLK activity in the neuron. In other embodiments, the biological sample comprises biological material selected from a neuron, neuronal cell lysate and DLK purified from a neuron.

In certain embodiments, the agent for use in the methods of the present invention inhibits or decreases the phosphorylation of a specific DLK amino acid residue such as the threonine at position 43 (T43) of SEQ ID NO:1 (human DLK); the threonine at position 43 of SEQ ID NO:2 (murine DLK); the serine at position 500 (S500) of SEQ ID NO:1 (human DLK); the serine at position 533 (S533) of SEQ ID NO:2 (murine DLK) or any combination thereof. In additional embodiments, the agent for use in the methods of the present invention inhibits phosphorylation of specific amino acid residues which are equivalent residues to the threonine at position 43 (T43) of SEQ ID NO:1 (human DLK); the threonine at position 43 of SEQ ID NO:2 (murine DLK); the serine at position 500 (S500) of SEQ ID NO:1 (human DLK); the serine at position 533 (S533) of SEQ ID NO:2 (murine DLK) in DLK from other species or isoforms and any combinations thereof.

In other embodiments, the agent for use in the methods of the present invention is selected from an antibody, a small molecule, a polypeptide, and a short interfering RNA (siRNA). In certain embodiments, the agent is an antibody. In specific embodiments, the antibody is selected from a polyclonal antibody, monoclonal antibody, chimeric antibody, humanized antibody, Fv fragment, Fab fragment, Fab′ fragment, and F(ab′)₂ fragment.

In further embodiments, the agent for use in the methods of the present invention is administered to a neuron or portion thereof. In certain embodiments, the neuron or portion thereof is present in a human subject, in a nerve graft or a nerve transplant, or is ex vivo or in vitro.

In additional embodiments, the agent for use in the methods of the present invention is a JNK inhibitor and/or the method further comprises the administration of an additional agent which is a JNK inhibitor. In specific embodiments, the JNK inhibitor inhibits JNK1, JNK2, JNK3 or any combination of JNK1, JNK2 and JNK3. In certain embodiments, the JNK inhibitor for use in the methods of the present invention inhibits JNK1, JNK2 and JNK3; or JNK1 and JNK2; or JNK1 and JNK3; or JNK2 and JNK3. In specific embodiments, the JNK inhibitor is selected from JNK Inhibitor V, JNK Inhibitor VII (TAT-TI-JIP₁₅₃₋₁₆₃), JNK Inhibitor VIII and siRNA, which inhibits expression of JNK polypeptides.

In additional embodiments, the patient being treated is suffering from a disease or condition selected from Alzheimer's Disease, Parkinson's disease, Parkinson's-plus diseases, amyotrophic lateral sclerosis (ALS), trigeminal neuralgia, glossopharyngeal neuralgia, Bell's Palsy, myasthenia gravis, muscular dystrophy, progressive muscular atrophy, primary lateral sclerosis (PLS), pseudobulbar palsy, progressive bulbar palsy, spinal muscular atrophy, inherited muscular atrophy, invertebrate disk syndromes, cervical spondylosis, plexus disorders, thoracic outlet destruction syndromes, peripheral neuropathies, prophyria, Huntington's disease, multiple system atrophy, progressive supranuclear palsy, corticobasal degeneration, dementia with Lewy bodies, frontotemporal dementia, demyelinating diseases, Guillain-Barré syndrome, multiple sclerosis, Charcot-Marie-Tooth disease, prion disease, Creutzfeldt-Jakob disease, Gerstmann-Sträussler-Scheinker syndrome (GSS), fatal familial insomnia (FFI), bovine spongiform encephalopathy, Pick's disease, epilepsy, and AIDS demential complex, chronic pain, fibromyalgia, spinal pain, carpel tunnel syndrome, pain from cancer, arthritis, sciatica, headaches, pain from surgery, muscle spasms, back pain, visceral pain, pain from injury, dental pain, neuralgia, such as neuogenic or neuropathic pain, nerve inflammation or damage, shingles, herniated disc, torn ligament, and diabetes, peripheral neuropathy or neuralgia caused by diabetes, cancer, AIDS, hepatitis, kidney dysfunction, Colorado tick fever, diphtheria, HIV infection, leprosy, lyme disease, polyarteritis nodosa, rheumatoid arthritis, sarcoidosis, Sjogren syndrome, syphilis, systemic lupus erythematosus, or oramyloidosis, nerve damage caused by exposure to toxic compounds, heavy metals, industrial solvents, drugs, chemotherapeutic agents, dapsone, HIV medications, cholesterol lowering drugs, heart or blood pressure medications, or ormetronidazole, injury to the nervous system caused by physical, mechanical, or chemical trauma, schizophrenia, delusional disorder, schizoaffective disorder, schizopheniform, shared psychotic disorder, psychosis, paranoid personality disorder, schizoid personality disorder, borderline personality disorder, anti-social personality disorder, narcissistic personality disorder, obsessive-compulsive disorder, delirium, dementia, mood disorders, bipolar disorder, depression, stress disorder, panic disorder, agoraphobia, social phobia, post-traumatic stress disorder, anxiety disorder, and impulse control disorders, glaucoma, lattice dystrophy, retinitis pigmentosa, age-related macular degeneration (AMD), photoreceptor degeneration associated with wet or dry AMD, other retinal degeneration, optic nerve drusen, optic neuropathy, and optic neuritis.

### BRIEF DESCRIPTION OF THE FIGURES

FIG. 1 DLK protein levels and molecular weight increase in response to neuronal stress in both in vitro and in vivo stress paradigms. (a) Embryonic dorsal root ganglion (DRG) neurons were cultured for 5 days in the presence of nerve growth factor (NGF) and were then deprived of NGF for 3 hours in four separate trials. In response, DLK quantity increases and this increase is accompanied by an upward mobility shift of DLK. cJun phosphorylation (p-cJun) occurs as a downstream consequence of DLK activation. (b) Retinas whose optic nerves had been crushed and their uncrushed contralateral controls were collected 3 days post-surgery. DLK levels and molecular weight in crushed retinas increase by 3 days following retina nerve crush. (c) Diagram of the retina nerve crush model showing location of the retina, crush site, proximal nerve, and distal nerve. (d) Within the crushed nerve, only DLK in the proximal nerve undergoes the stress-dependent increase in molecular weight and amount observed in whole retinas after nerve crush. Nerve crush was performed on mice of the given genotypes and nerves were collected 24 hours later. WT: C57BL/6. Cre−: DLK^{lox}/DLK^{lox}; Cre−. Cre+: DLK^{lox}/DLK^{lox}; Cre+. The shift in mobility can be observed following crush (red arrows) in the proximal nerve. *: a background band observed in nerve lysates blotted for DLK. (e) Mean ratio of DLK protein quantity in −NGF vs. +NGF samples shown in (a) and in crushed vs. uncrushed samples shown in (b). In each stress paradigm, the quantity of DLK was measured in ImageLab and normalized to the actin loading control. In NGF withdrawal samples, the ratio of DLK in −NGF and +NGF was taken for each adjacent set of conditions. In retina nerve crush, the ratio of DLK quantity in crushed and uncrushed eyes was taken for each of four mice. TF withdrawal: mean=2.12±0.127. Nerve crush: mean=1.497±0.157. *P=0.02 by Mann-Whitney U test comparing the means to 1. (f) Mean molecular weight (MW) of DLK in +NGF and −NGF samples. Molecular weight was calculated in Image Lab using the molecular weight analysis tool comparing the molecular weights of DLK to those of a known set of molecular weight standards. +NGF: Mean=115.1±0.7723. −NGF: Mean=120.0±0.8050. *P=0.03 by Mann-Whitney U test. (g) Lambda protein phosphatase (λpp) treatment of DRG lysates equalizes DLK molecular weight in −NGF and +NGF conditions. All error bars are standard error of the mean.

FIG. 2 DLK protein is stabilized in response to trophic factor withdrawal in embryonic sensory neurons. (a) Measurement of relative amounts of DLK transcript by qRT-PCR in −NGF vs. +NGF culture conditions (blue bar) or in crushed vs. uncrushed retinas from 3 replicate mice (black bars). Error bars are standard deviations based on three technical replicates. −NGF vs +NGF: 1.036±0.0234. mouse 1: 0.995±0.0315. mouse 2: 0.970±0.0462. mouse 3: 0989±0.040. (b) 8-hour timecourse of trophic factor withdrawal and +NGF controls in the presence of 5 μM cycloheximide. (c) Quantification of three repeated trials of experiment performed in (b). Bands at each time point were quantified relative to actin loading controls, and the mean band intensity at each time point was calculated and divided by the intensity at t=0. Plotted is the relative amount of DLK remaining at a given time compared to the amount at time 0. Error bars are standard deviations. Each timecourse was fit to a line in GraphPad Prism software. *P=0.0109 when comparing the slopes of the two lines with ANCOVA. (d) DRGs were treated with the given conditions for three hours and lysed. OA: 200 nM okadaic acid. MG132 was used at 30 μM.

FIG. 3 The ubiquitin-proteasome system regulates DLK levels in a stress-dependent manner. (a) DLK ubiquitination is reduced by trophic factor withdrawal. NGF-deprived and control DRGs were collected and lysed after 3 hours of treatment. Ubiquitinated proteins were immunoprecipitated from the lysates and immunoprecipitates were blotted for DLK and ubiquitin. Mouse IgG controls were used as negative controls to demonstrate antibody specificity. (b) Western blots for DLK, USP9X, p-cJun, and tubulin following NGF withdrawal in USP9X loxp/loxp Cre− and Cre+ embryonic DRGs. (c) Quantification of blots for DLK and USP9X shown in (b). Loss of ˜95% of USP9X (left panel) results in an overall decrease in DLK levels but does not alter the relative levels of DLK in the + and −NGF conditions (right panel-compare fold change in Cre+ to that in Cre−). (d) DLK protein levels in the +NGF condition are elevated in Phr1^{mag} loss-of-function mutants compared to Phr1^{WT}. Despite this increase, downstream activation of p-cJun is unaffected in the Phr1^{mag} mutant. (e) Quantification of DLK blot shown in (d) normalized to Tuj loading control. (f) Immunoprecipitation of ubiquitinated proteins shows that DLK is less ubiquitinated in Phr1^{mag/mag} homozygotes than in Phr^{WT/mag} heterozygote controls. Left panel: Immunoprecipitation with anti-ubiquitin (“Ub”) followed by blotting for DLK. Immunoprecipitation with mouse IgG was used as a control for antibody specificity. Bracket: highlights the main difference seen between Phr1^{mag} heterozygotes and homozygotes: a lack of polyubiquitinated DLK in the knockouts. Right panel: Blots of the input lysates for DLK and tubulin. Despite the fact that more DLK was present in the input, less DLK was pulled down by the anti-ubiquitin antibody.

FIG. 4 DLK stabilization depends on DLK activity and on downstream targets of DLK. (a) Transient transfection of HEK 293T cells shows that a kinase dead version of DLK (S302A) is expressed at lower levels than wild type DLK. Co-transfection with USP9X rescues this effect. (b) Co-transfection of DLK with a dominant negative DLK Leucine Zipper domain construct N-terminally tagged with myc epitope (myc-DLK-LZ) decreases DLK expression compared to co-transfection with a GFP expression construct. (c) Inhibition of JNK activity with two different JNK inhibitors (JNK8 and JNK7, both at 10 μM) in a stable cell with a dox-inducible DLK expression construct reduces DLK expression. (d) Knockdown of JNK3 in a JNK2 KO background blocks the increase in DLK quantity observed with NGF withdrawal in embryonic DRGs. (e) At 18 hours following nerve crush, JNK2/3 double knockout retinas do not have activated DLK as assayed by the higher molecular form (arrow), seen in crushed retinas from littermate controls.

FIG. 5 Identification of phosphorylation sites in murine DLK whose phosphorylation state is modulated by DLK or JNK activity. (a) Expression of DLK in 293T cells in the heavy and light SILAC conditions used for mass spectrometry. After expression, Flag-tagged DLK was immunoprecipitated (IP) and IPs of the given conditions were combined for ratiometric analysis of phosphorylation sites in DLK. WT DLK: wild type DLK. DLK^{S302A}: kinase dead point mutant. CA-JNK: constitutively active JNK construct (see methods). JNKi: JNK inhibitor 8, 10 μM. OA: okadaic acid, 200 nM. (b) Schematic of DLK domains and locations of noted phosphorylation sites. Numbering is based on murine DLK. N-term: N-terminal domain of DLK. Kinase domain: catalytic DLK domain. LZ: leucine zipper motifs. C-term: C-terminal domain. Domains are arranged according to reference Holzman, L. B., Merritt, S. E. & Fan, G. The Journal of biological chemistry 269, 30808-30817 (1994). (c) Summary of identified phosphorylation site changes. Sites listed showed the largest effects and consistency across conditions, or in the case of 5295-T306, are known sites within the kinase activation loop. ∞: phosphorylation of this site was observed in condition A but not in condition B. N/A: phosphorylation of this site was not observed in either condition. Numbers given are the fold changes in phosphorylation of the site in the condition A vs. condition B, and up or down arrows denote the direction of change (e.g. For top-right box, there is 7.78-fold more phosphorylation of T43 in okadaic-acid-treated cells expressing DLK than in cells expressing DLK with no okadaic acid). *: This site contains a threonine (T) or serine (S) followed by a proline. A flanking proline is found in the vast majority of MAPK phosphorylation sites. The column entitled “Fits with JNK hypothesis?” summarizes whether the pattern of phosphorylation across the four conditions fits with the hypothesis that phosphorylation of this site by JNK occurs and is responsible for stabilization of DLK. Check mark: pattern of phosphorylation is consistent with this hypothesis. ˜: pattern of phosphorylation is partially consistent with this hypothesis. X: pattern of phosphorylation is inconsistent with this hypothesis. For 5643, the Ascore of 10.3 favors the preceding serine with ˜90% confidence. Phosphorylation sites with Ascores of less than 13 are generally considered ambiguously localized, but because S643 is the last of four consecutive serines and resides immediately adjacent to proline, we conclude that phosphorylation occurs on this site and not S642. For the last row (S295-T306), two phosphorylated residues were detected on this peptide, but because of a lack of site determining ions a single, doubly-modified sequence cannot be unambiguously assigned. Given the number of possible permutations, it is possible that several multiply-phosphopeptide sequences occur. For this reason, data for this doubly modified sequence are shown in the aggregate, rather than specifically by site.

FIG. 6 Identified sites are phosphorylated after neuronal stress (a) Western blots on lysates from HEK 293T cells in which wild type DLK (DLK^{WT}) and the given phosphoincompetent point mutants were transiently expressed. p-T43, p-S272, p-S533: blots with phospho-specific antibodies for each of these sites. (b) DLK^{T43} and DLK^{S533} can be directly phosphorylated by JNK. Purified DLK^{S302A} was incubated with (right lane) or without (left lane) purified JNK3 and blotted with the shown phospho-specific antibodies. (c) Western blots on lysates from trophic-factor deprived sensory neurons showed anti-phosphoT43 immunoreactivity appearing in the −NGF condition. Treatment with lambda protein phosphatase demonstrates the specificity of the antibody for phosphorylated protein. (d) Blotting trophic-factor-deprived DRGs from DLK loxp/loxp Cre− and Cre+ embryos shows that the anti-p-T43 antibody is specific for DLK. (e) Immunoprecipitation of DLK from crushed retina lysates and uncrushed controls followed by blotting with phospho-T43 and phospho-S533-specific antibodies shows that these sites are phosphorylated following optic nerve crush. Left panel: input to immunoprecipitations showing the increase in DLK levels and phosphorylation (apparent molecular weight shift) with optic nerve crush. Right panel: immunoprecipitation from crushed lysates or uncrushed controls with anti-DLK or a control rabbit IgG followed by blotting with the given antibodies.

FIG. 7 DLK modulates downstream pro-apoptotic signaling in a dose-dependent manner. (a) A timecourse of trophic factor withdrawal in DLK+/+ (WT) and DLK+/− (het) DRGs reveals that the reduction of DLK protein levels in heterozygous neurons results in reduced downstream activation of JNK and cJun. (b, d) Staining of nerve-crushed retinas in DLK+/+ and DLK+/− mice, and (c, e, f) quantifications of stainings shown. (b,c) p-cJun 6 hours post-crush. Quantification shown is the mean number of p-cJun positive cells per retina. *P=0.0014 by student's t test. Mean of WT=2291±299. Mean of het=689±97.6. n=7 WT and 5 het animals. (d) caspase-3 and Brn3 staining 3 days post-crush (e) Quantification of mean number of caspase-3-positive cells per retina. *P=0.0001 by student's t test. Mean of WT=823±36. Mean of het=79±20. n=4 WT and 3 het animals. (f) Quantification of ratio of Brn3-positive cells per retina in crushed vs. uncrushed retinas. *P=0.0253 by student's t test. Mean of WT=0.27±0.086. Mean of het=0.56±0.017. Error bars are standard error of the mean. n=5 WT and 4 het animals. Scale bars=100 μm.

FIG. 8 Observation of DLK gel mobility shift with neuronal stress. (a) Blots of 3-day crushed retina lysates from wild type (WT) and DLK^{loxp/loxp} (loxp) mice injected with AAV-Cre virus. The DLK gel mobility shift is seen in wild type retinas in the form of a doublet (red arrows), but this is not observed in loxp mice. Because the loxp mice have only recombined Dlk in retinal ganglion cells, this shows that the upper band (red arrow) that appears with retina nerve crush is due to phosphorylation of DLK in retinal ganglion cells specifically. (b) Difference in mobility of DLK in SDS-PAGE with two different running buffers. MOPS buffer: SDS-PAGE running solution containing MOPS as a buffer. MES buffer: SDS-PAGE running solution containing MES as a buffer.

FIG. 9 USP9X activity does not change with trophic factor withdrawal. Incubation of + and −NGF-treated DRGs with HA-tagged ubiquitin vinyl sulfones, which covalently bind to the active site of deubiquitinating enzymes (DUBs), shows no change in USP9X activity with trophic factor withdrawal. N-ethylmaleimide (NEM), which inhibits DUBs is used as a negative control.

FIG. 10 No difference in the timecourse of degeneration of sensory neuron axons following NGF withdrawal in Phr1 KO neurons. Embryonic DRGs were cultured from wild type or Phr1 knockout littermate embryos and deprived of NGF after 3 days in vitro. 18 hours later, both cultures were equally degenerated, as visualized by tubulin staining.

FIG. 11 Characterization of markers of cell viability, intact axonal structure, and activation of neuronal stress signaling in retinas following optic nerve crush. (a) Stainings for Brn3, γ-synuclein, and neurofilament-M (NF-M) at two weeks post-crush, compared to uncrushed wild type controls. (b) p-cJun staining in wild type and DLK heterozygous retinas 24 hours post-surgery. No significant difference between wild type and heterozygous retinas in p-cJun staining is observed at this time point.

FIG. 12 Blots for total JNK, JNK2, and JNK3 on retina lysate samples. Loss of immunoreactivity is seen in the JNK2/3 double knockout.

### DETAILED DESCRIPTION OF EMBODIMENTS OF THE INVENTION

### I. Definitions

The term “dual leucine zipper kinase” or “DLK” as used herein, refers to any native DLK from any vertebrate source, including mammals such as primates (e.g. humans) and rodents (e.g., mice and rats), unless otherwise indicated. The term encompasses “full-length”, unprocessed DLK as well as any form of DLK that results from processing in the cell including the various polypeptide isoforms encoded by DLK pre-mRNA, naturally occurring variants of DLK, (e.g., splice variants or allelic variants) and post-translationally modified processed forms of DLK known in the art. One such variant example of DLK includes Isoform 2 (UniProtKB/Swiss-Prot Accession No. Q12852-2). DLK is also known by the names “mitogen-activated protein kinase kinase kinase 12”, “MAP3K12”, “dual leucine zipper bearing kinase”, “leucine zipper protein kinase” (ZPK), and “MAPK-upstream kinase” (MUK). Human DLK is 859 amino acids in length as described in UniProtKB/Swiss-Prot Accession No. Q12852, and is incorporated by reference herein. An exemplary amino acid sequence for human DLK is as follows:

(SEQ ID NO: 1)
MACLHETRTPSPSFGGFVSTLSEASMRKLDPDTSDCTPEKDLTPTHVLQL
HEQDAGGPGGAAGSPESRASRVRADEVRLQCQSGSGFLEGLFGCLRPVWT
MIGKAYSTEHKQQQEDLWEVPFEEILDLQWVGSGAQGAVFLGRFHGEEVA
VKKVRDLKETDIKHLRKLKHPNIITFKGVCTQAPCYCILMEFCAQGQLYE
VLRAGRPVTPSLLVDWSMGIAGGMNYLHLHKIIHRDLKSP NMLITYDDV
VKISDFGTSKELSDKSTKMSFAGTVAWMAPEVIRNEPVSEKVDIWSFGVV
LWELLTGEIPYKDVDSSAIIWGVGSNSLHLPVPSSCPDGFKILLRQCWNS
KPRNRPSFRQILLHLDIASADVLSTPQETYFKSQAEWREEVKLHFEKIKS
EGTCLHRLEEELVMRRREELRHALDIREHYERKLERANNLYMELNALMLQ
LELKERELLRREQALERRCPGLLKPHPSRGLLHGNTMEKLIKKRNVPQKL
SPHSKRPDILKTESLLPKLDAALSGVGLPGCPKGPPSPGRSRRGKTRHRK
ASAKGSCGDLPGLRTAVPPHEPGGPGSPGGLGGGPSAWEACPPALRGLHH
DLLLRKMSSSSPDLLSAALGSRGRGATGGAGDPGSPPPARGDTPPSEGSA
PGSTSPDSPGGAKGEPPPPVGPGEGVGLLGTGREGTSGRGGSRAGSQHLT
PAALLYRAAVTRSQKRGISSEEEEGEVDSEVELTSSQRWPQSLNMRQSLS
TFSSENPSDGEEGTASEPSPSGTPEVGSTNTDERPDERSDDMCSQGSEIP
LDPPPSEVIPGPEPSSLPIPHQELLRERGPPNSEDSDCDSTELDNSNSVD
ALRPPASLPP.

Murine DLK is 888 amino acids in length as described in UniProtKB/Swiss-Prot Accession No. Q60700, and is incorporated by reference herein. An exemplary amino acid sequence for murine DLK is as follows:

(SEQ ID NO: 2)
MACLHETRTPSPSFGGFVSTLSEASMRKLDPDTSDCTPEKDLTPTQCVLRD
VVPLGGQGGGGPSPSPGGEPPPEPFANSVLQLHEQDTGGPGGATGSPESRA
SRVRADEVRLQCQSGSGFLEGLFGCLRPVWTMIGKAYSTEHKQQQEDLWEV
PFEEILDLQWVGSGAQGAVFLGRFHGEEVAVKKVRDLKETDIKHLRKLKHP
NIITFKGVCTQAPCYCILMEFCAQGQLYEVLRAGRPVTPSLLVDWSMGIAG
GMNYLHLHKIIHRDLKSPNMLITYDDVVKISDFGTSKELSDKSTKMSFAGT
VAWMAPEVIRNEPVSEKVDIWSFGVVLWELLTGEIPYKDVDSSAIIWGVGS
NSLHLPVPSSCPDGFKILLRQCWNSKPRNRPSFRQILLHLDIASADVLSTP
QETYFKSQAEWREEVKLHFEKIKSEGTCLHRLEEELVMRRREELRHALDIR
EHYERKLERANNLYMELNALMLQLELKERELLRREQALERRCPGLLKSHPS
RGLLHGNTMEKLIKKRNVPQKLSPHSKRPDILKTESLLPKLDAALSGVGLP
GCPKGPPSPGRSRRGKTRHRKASAKGSCGDLPGLRAALPPHEPGGLGSPGG
LGVGPSAWDACPPALRGLHHDLLLRKMSSSSPDLLSAALGARGRGATGGAR
DPGSPPPPQGDTPPSEGSAPGSTSPDSPGGAKGEPPPPVGPGEGVGLLGTG
REGTAGRGGNRAGSQHLTPAALLYRAAVTRSQKRGISSEEEEGEVDSEVEL
PPSQRWPQGPNMRQSLSTFSSENPSDVEEGTASEPSPSGTPEVGSTNTDER
PDERSDDMCSQGSEIPLDLPTSEVVPEREASSLPMQHQDGQGPNPEDSDCD
STELDNSNSIDALRPPASLPP.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g. scFv); and multispecific antibodies formed from antibody fragments.

An “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more. An exemplary competition assay is provided herein.

The term “chimeric” antibody refers to an antibody in which a portion of the heavy and/or light chain is derived from a particular source or species, while the remainder of the heavy and/or light chain is derived from a different source or species.

An “effective amount” of an agent, e.g., a pharmaceutical formulation, refers to an amount effective, at dosages and for periods of time necessary, to achieve the desired therapeutic or prophylactic result.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody.

A humanized antibody optionally may comprise at least a portion of an antibody constant region derived from a human antibody. A “humanized form” of an antibody, e.g., a non-human antibody, refers to an antibody that has undergone humanization.

An “individual” or “patient” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or patient or subject is a human.

An “isolated” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr. B 848:79-87 (2007).

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

The term “package insert” is used to refer to instructions customarily included in commercial packages of therapeutic products, that contain information about the indications, usage, dosage, administration, combination therapy, contraindications and/or warnings concerning the use of such therapeutic products.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject., A pharmaceutically acceptable carrier includes, but is not limited to, a buffer, excipient, stabilizer, or preservative.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies of the invention are used to delay development of a disease or to slow the progression of a disease.

The term “short-interfering RNA (siRNA)” refers to small double-stranded RNAs that interfere with gene expression. siRNAs are mediators of RNA interference, the process by which double-stranded RNA silences homologous genes. siRNAs typically are comprised of two single-stranded RNAs of about 15-25 nucleotides in length that form a duplex, which may include single-stranded overhang(s). Processing of the double-stranded RNA by an enzymatic complex, for example, polymerases, results in cleavage of the double-stranded RNA to produce siRNAs. The antisense strand of the siRNA is used by an RNA interference (RNAi) silencing complex to guide mRNA cleavage, thereby promoting mRNA degradation. To silence a specific gene using siRNAs, for example, in a mammalian cell, a base pairing region is selected to avoid chance complementarity to an unrelated mRNA. RNAi silencing complexes have been identified in the art, such as, for example, by Fire et al., Nature 391:806-81 (1998) and McManus et al., Nat. Rev. Genet. 3(10):737-747 (2002).

The term “interfering RNA (RNAi)” is used herein to refer to a double-stranded RNA that results in catalytic degradation of specific mRNAs, and thus can be used to inhibit/lower expression of a particular gene.

The phrases “preventing axon degeneration,” “preventing neuron degeneration,” “preventing neuronal degeneration,” “inhibiting neuronal degeneration,” “inhibiting axon degeneration,” or “inhibiting neuron degeneration” as used herein include (i) the ability to inhibit or prevent axon or neuron degeneration in patients newly diagnosed as having a neurodegenerative disease or disorder or at risk of developing a new neurodegenerative disease or disorder and (ii) the ability to inhibit or prevent further axon or neuron degeneration in patients who are already suffering from, or have symptoms of, a neurodegenerative disease or disorder. Preventing axon or neuron degeneration includes decreasing or inhibiting axon or neuron degeneration, which may be characterized by complete or partial inhibition of neuron or axon degeneration. This can be assessed, for example, by analysis of neurological function. The above-listed terms also include in vitro and ex vivo methods. Further, the phrases “preventing neuron degeneration” and “inhibiting neuron degeneration” include such inhibition with respect to the entire neuron or a portion thereof, such as the neuron cell body, axons, and dendrites. The administration of one or more agent as described herein may result in at least a 10% decrease (e.g., at least 15%, 20%, 25%, 30%, 35%, 40%, 45%, 50%, 55%, 60%, 65%, 70%, 75%, 80%, 85%, 90%, or even 100% decrease) in one or more (e.g., 1, 2, 3, 4, 5, 6, 7, 8, or 9) symptoms of a disorder of the nervous system; a condition of the nervous system that is secondary to a disease, condition, or therapy having a primary effect outside of the nervous system; an injury to the nervous system caused by physical, mechanical, or chemical trauma, pain; an ocular-related neurodegeneration; memory loss; or a psychiatric disorder (e.g., tremors, slowness of movement, ataxia, loss of balance, depression, decreased cognitive function, short-term memory loss, long-term memory loss, confusion, changes in personality, language difficulties, loss of sensory perception, sensitivity to touch, numbness in extremities, muscle weakness, muscle paralysis, muscle cramps, muscle spasms, significant changes in eating habits, excessive fear or worry, insomnia, delusions, hallucinations, fatigue, back pain, chest pain, digestive problems, headache, rapid heart rate, dizziness, blurred vision, shadows or missing areas of vision, metamorphopsia, impairment in color vision, decreased recovery of visual function after exposure to bright light, and loss in visual contrast sensitivity) in a subject or population compared to a control subject or population that does not receive the one or more agent described herein. The administration of one or more agent as described herein may result in at least a 10% decrease (e.g., at least 15%, 20%, 25%, 30%, 35%, 40%, 45%, 50%, 55%, 60%, 65%, 70%, 75%, 80%, 85%, 90%, 95%, or even 100% decrease) in the number of neurons (or neuron bodies, axons, or dendrites thereof) that degenerate in a neuron population or in a subject compared to the number of neurons (or neuron bodies, axons, or dendrites thereof) that degenerate in neuron population or in a subject that is not administered the one or more of the agents described herein. The administration of one or more agent as described herein may result in at least a 10% decrease (e.g., at least 15%, 20%, 25%, 30%, 35%, 40%, 45%, 50%, 55%, 60%, 65%, 70%, 75%, 80%, 85%, 90%, 95%, or even 100% decrease) in the likelihood of developing a disorder of the nervous system; a condition of the nervous system that is secondary to a disease, condition, or therapy having a primary effect outside of the nervous system; an injury to the nervous system caused by physical, mechanical, or chemical trauma, pain; an ocular-related neurodegeneration; memory loss; or a psychiatric disorder in a subject or a subject population compared to a control subject or population not treated with the one or more agent described herein.

The term “administering” as used herein refers to contacting a neuron or portion thereof with an agent as described herein. This includes administration of the agent to a subject in which the neuron or portion thereof is present, as well as introducing the agent into a medium in which a neuron or portion thereof is cultured.

The term “neuron” as used herein denotes nervous system cells that include a central cell body or soma, and two types of extensions or projections: dendrites, by which, in general, the majority of neuronal signals are conveyed to the cell body, and axons, by which, in general, the majority of neuronal signals are conveyed from the cell body to effector cells, such as target neurons or muscle. Neurons can convey information from tissues and organs into the central nervous system (afferent or sensory neurons) and transmit signals from the central nervous systems to effector cells (efferent or motor neurons). Other neurons, designated interneurons, connect neurons within the central nervous system (the brain and spinal column). Certain specific examples of neuron types that may be subject to treatment according to the invention include cerebellar granule neurons, dorsal root ganglion neurons retinal ganglion cells, retinal optic nerves and cortical neurons.

Administration “in combination with” one or more further therapeutic agents includes simultaneous (concurrent) and consecutive administration, in any order.

As used herein, the term “neuronal stress” means the application of a stress to a neuron such as, but not limited to, disease, injury, ischemia, excitotoxicity, axon transection, UV irradiation, stimulation by cytokines, ceramide exposure, or the absence of nerve growth factor. The neuronal stress may result in neuronal degeneration and cell death, including by activation of an apoptotic signaling cascade in the neuron.

The term “stress dependent DLK activity,” as used herein, means the activation of DLK in response to a neuronal stress.

The term “pro-apopototic DLK activity,” as used herein, means the activation of DLK which would favor or induce an apoptotic signaling cascade in a neuron.

### II. Methods

In one aspect, the invention is based, in part, on the discovery that DLK is phosphorylated in response to stress or injury in neurons. The increase in DLK phosphorylation results in stabilization of DLK and an increase in DLK protein levels in the injured or stressed neuron. Certain amino acid residues in DLK are phosphorylated in response to neuronal stress or injury and are important for increased DLK stability and ultimately stress-dependent or pro-apoptotic activity of DLK necessary for axon degeneration and neuronal apoptosis.

Thus, the invention includes methods of preventing or inhibiting neuronal degeneration by use of an agent which inhibits or decreases the phosphorylation of DLK, thus decreasing DLK stability. Additionally, the invention includes methods of inhibiting or decreasing phosphorylation of certain amino acid residues in DLK thereby decreasing the stability of DLK. In other aspects, the invention includes methods for decreasing DLK stability in a neuron, the method comprising administering to a neuron or portion thereof, an agent which inhibits or reduces the phosphorylation of DLK and in certain instances, the agent inhibits phosphorylation of certain amino acid residues of DLK, wherein the decrease in phosphorylation results in a decrease in DLK stability.

The invention also includes methods for detecting stress dependent or pro-apopototic activity in a neuron, the method comprising contacting a biological sample with an antibody which specifically recognizes a phosphorylated form of DLK and detecting the binding of said antibody to the phosphorylated form of DLK, wherein binding of the antibody to the phosphorylated form of DLK indicates or is indicative of stress dependent or pro-apoptotic DLK activity.

The neuron or portion thereof used in the methods of the present invention include neurons selected from the group consisting of a cerebellar granule neuron, a dorsal root ganglion neuron, a cortical neuron, a sympathetic neuron, a retinal ganglion cell, a retina optic nerve and a hippocampal neuron.

The agents used in the methods of the present invention include inhibitors of DLK phosphorylation. Further, the agents for use in the methods of the invention are selected, for example, from the group consisting of antibodies, polypeptides, peptides, peptibodies, nucleic acid molecules, short interfering RNAs (siRNAs), polynucleotides, aptamers, small molecules, and polysaccharides. In the case of antibodies, the antibodies are selected from monoclonal antibodies, chimeric antibodies, humanized antibodies, human antibodies, or antibody fragments (e.g., an Fv, Fab, Fab′, or F(ab′)₂ fragment).

Additional agents for use in the methods of the present invention include inhibitors of proteins which phosphorylate DLK such as JNK. Non-limiting examples include inhibitors of JNK1, JNK2 and/or JNK3. Additional examples include inhibitors of JNK1 and JNK2; JNK1 and JNK3; JNK1 and JNK3; JNK2 and JNK3; and JNK1, JNK2 and JNK3. Such JNK inhibitors include but are not limited to JNK Inhibitor V, JNK Inhibitor VII (TAT-TI-JIP₁₅₃₋₁₆₃), JNK Inhibitor VIII, SC-202673, SY-CC-401, SP600125, AS601245, and XG-102, as well as Catalog Nos. 420119, 420130, 420131, 420123, 420116, 420118, 420136, 420129, 420135, 420134, 420133, 420140, and 420128 from EMD Biosciences and siRNA which inhibit the expression of JNK1, JNK2, JNK3 or any combination thereof. For example a siRNA sequences targeting various JNKs include the JNK1 sequence of TTGGATGAAGCCATTAGACTA (SEQ ID NO:3)), the JNK2 sequence of ACCTTTAATGGACAA CATTAA (SEQ ID NO:4) or AAGGATTAGCTTTGTATCATA (SEQ ID NO:5)), and the JNK3 sequence of CCCGCATGTGTCT GTATTCAA (SEQ ID NO:6)).

In certain embodiments, the neuron or portion thereof in the methods of the invention is present in a subject, such as a human subject. The subject, for example, is developing or is at risk of developing a disease or condition selected from the group consisting of (i) disorders of the nervous system, (ii) conditions of the nervous system that are secondary to a disease, condition, or therapy having a primary effect outside of the nervous system, (iii) injuries to the nervous system caused by physical, mechanical, or chemical trauma, (iv) pain, (v) ocular-related neurodegeneration, (vi) memory loss, and (vii) psychiatric disorders.

Examples of disorders of the nervous system include amyotrophic lateral sclerosis (ALS), trigeminal neuralgia, glossopharyngeal neuralgia, Bell's Palsy, myasthenia gravis, muscular dystrophy, progressive muscular atrophy, primary lateral sclerosis (PLS), pseudobulbar palsy, progressive bulbar palsy, spinal muscular atrophy, inherited muscular atrophy, invertebrate disk syndromes, cervical spondylosis, plexus disorders, thoracic outlet destruction syndromes, peripheral neuropathies, prophyria, Alzheimer's disease, Huntington's disease, Parkinson's disease, Parkinson's-plus diseases, multiple system atrophy, progressive supranuclear palsy, corticobasal degeneration, dementia with Lewy bodies, frontotemporal dementia, demyelinating diseases, Guillain-Barre syndrome, multiple sclerosis, Charcot-Marie-Tooth disease, prion disease, Creutzfeldt-Jakob disease, Gerstmann-Straussler-Scheinker syndrome (GSS), fatal familial insomnia (FFI), bovine spongiform encephalopathy, Pick's disease, epilepsy, and AIDS demential complex.

Examples of pain include chronic pain, fibromyalgia, spinal pain, carpel tunnel syndrome, pain from cancer, arthritis, sciatica, headaches, pain from surgery, muscle spasms, back pain, visceral pain, pain from injury, dental pain, neuralgia, such as neuogenic or neuropathic pain, nerve inflammation or damage, shingles, herniated disc, torn ligament, and diabetes.

Examples of conditions of the nervous system that are secondary to a disease, condition, or therapy having a primary effect outside of the nervous system include peripheral neuropathy or neuralgia caused by diabetes, cancer, AIDS, hepatitis, kidney dysfunction, Colorado tick fever, diphtheria, HIV infection, leprosy, lyme disease, polyarteritis nodosa, rheumatoid arthritis, sarcoidosis, Sjogren syndrome, syphilis, systemic lupus erythematosus, and amyloidosis.

Examples of injuries to the nervous system caused by physical, mechanical, or chemical trauma include nerve damage caused by exposure to toxic compounds, heavy metals, industrial solvents, drugs, chemotherapeutic agents, dapsone, HIV medications, cholesterol lowering drugs, heart or blood pressure medications, and metronidazole. Additional examples include burn, wound, surgery, accidents, ischemia, prolonged exposure to cold temperature, stroke, intracranial hemorrhage, and cerebral hemorrhage.

Examples of psychiatric disorders include schizophrenia, delusional disorder, schizoaffective disorder, schizopheniform, shared psychotic disorder, psychosis, paranoid personality disorder, schizoid personality disorder, borderline personality disorder, anti-social personality disorder, narcissistic personality disorder, obsessive-compulsive disorder, delirium, dementia, mood disorders, bipolar disorder, depression, stress disorder, panic disorder, agoraphobia, social phobia, post-traumatic stress disorder, anxiety disorder, and impulse control disorders.

Examples of ocular-related neurodegeneration include glaucoma, lattice dystrophy, retinitis pigmentosa, age-related macular degeneration (AMD), photoreceptor degeneration associated with wet or dry AMD, other retinal degeneration, optic nerve drusen, optic neuropathy, and optic neuritis. Examples of glaucoma include primary glaucoma, low-tension glaucoma, primary angle-closure glaucoma, acute angle-closure glaucoma, chronic angle-closure glaucoma, intermittent angle-closure glaucoma, chronic open-angle closure glaucoma, pigmentary glaucoma, exfoliation glaucoma, developmental glaucoma, secondary glaucoma, phacogenic glaucoma, glaucoma secondary to intraocular hemorrhage, traumatic glaucoma, neovascular glaucoma, drug-induced glaucoma, toxic glaucoma, and glaucoma associated with intraocular tumors, retinal detachments, severe chemical burns of the eye, and iris atrophy.

In certain embodiments, contacting the neuron or portion thereof with the agent, according to the methods of the invention, involves administering to a subject a pharmaceutical composition including the agent. The administering is carried out by, for example, intravenous infusion; injection by intravenous, intraperitoneal, intracerebral, intramuscular, intraocular, intraarterial or intralesional routes; or topical or ocular application. Further, the methods of the invention includes administering to a subject one or more additional pharmaceutical agents.

In other examples, the neuron or portion thereof treated according to the methods of the invention is ex vivo or in vitro (e.g., a nerve graft or nerve transplant).

The invention also includes methods of identifying agents for use in inhibiting degeneration of a neuron or a portion thereof. These methods involve contacting a neuron or portion thereof with a candidate agent in an assay of axon or neuron degeneration (e.g., anti-nerve growth factor (NGF) antibodies, serum deprivation/KCl reduction, retina optic nerve crush and/or rotenone treatment). Detection of reduced degeneration of the neuron or portion thereof in the presence of the candidate agent, relative to a control, indicates the identification of an agent for use in inhibiting degeneration of a neuron or portion thereof. The candidate agent is, for example, selected from the group consisting of antibodies, polypeptides, peptides, peptibodies, nucleic acid molecules, short interfering RNAs (siRNAs), polynucleotides, aptamers, small molecules, and polysaccharides.

A. Screening Assays to Identify and Characterize Agents for Use in the Methods of the Invention

The invention is based in part on the discovery that stress-induced or neuron injury induced phosphorylation of DLK results in DLK stability and ultimately pro-apoptotic DLK activity. The invention includes methods of inhibiting and/or preventing neuron or axon degeneration by use of agents which inhibit or decrease DLK phosphorylation as described herein. As described herein, the methods are carried out in vivo, such as in the treatment of neurological disorders or injuries to the nervous system. The methods are carried out in vitro or ex vivo, such as in laboratory studies of neuron function and in the treatment of nerve grafts or transplants.

Agents for use in the methods of the invention are described herein. Additional agents for use in the invention can be identified using standard screening methods summarized below.

In additions to the agents described here, additional agents for use in the methods of the present invention which inhibit or reduce phosphorylation of DLK and inhibit or prevent neuronal degeneration can be screened using the following assays or combination of assays. In addition to the assays described below, for which the read-out is phosphorylation of DLK or inhibition of neuron or axon degeneration, the invention also employs assays directed at detecting agents which simply bind or detect certain phospho-forms of DLK. Thus, the invention includes the use of screening assays which identify compounds that bind or complex with specific phospho-forms of DLK.

In binding assays, the interaction is binding, and the complex formed can be isolated or detected in the reaction mixture. In a particular embodiment, either the target polypeptide or the agent candidate is immobilized on a solid phase, e.g., on a microtiter plate, by covalent or non-covalent attachments. Non-covalent attachment generally is accomplished by coating the solid surface with a solution of the polypeptide and drying. Alternatively, an immobilized antibody, e.g., a monoclonal antibody, specific for the target polypeptide to be immobilized can be used to anchor it to a solid surface. The assay is performed by adding the non-immobilized component, which may be labeled by a detectable label, to the immobilized component, e.g., the coated surface containing the anchored component. When the reaction is complete, the non-reacted components are removed, e.g., by washing, and complexes anchored on the solid surface are detected. When the originally non-immobilized component carries a detectable label, the detection of label immobilized on the surface indicates that complexing occurred. Where the originally non-immobilized component does not carry a label, complexing can be detected, for example, by using a labeled antibody specifically binding the immobilized complex.

Additionally, assays for measuring the impact of a candidate agent on the activity of a protein kinase are known in the art, and include direct phosphorylation assays, typically interpreted via radio-labeled phosphate, phosphorylation-specific antibodies to a substrate, and cell-based assays that measure the downstream consequence of kinase activity, e.g., activation of a reporter gene. Both of these major strategies, in addition to alternative assays based on fluorescence polarization, may be used in small-scale or high-throughput format to identify, validate, or characterize an inhibitor (see, for example, Favata et al., J. Biol. Chem. 273:18623-18632, 1998; Parker et al., J. Biomol. Screening 5:77-99, 2000; Singh et al., Comb. Chem. High Throughput Screen 8:319-325, 2005; Garton et al., Meth. Enz. 439:491-500, 2008; and Kupchko et al., Anal. Biochem. 317:210-217, 2003).

The screening assays specifically discussed herein are for the purpose of illustration only. A variety of other assays, which can be selected depending on the particular target and type of agent (e.g., antibodies, polypeptides, peptides, non-peptide small organic molecules, nucleic acid molecules, etc.) are well known to those skilled in the art and may also be used in the present invention.

The assays described herein may also be used to screen libraries of compounds including, without limitation, chemical libraries, natural product libraries (e.g., collections of microorganisms, animals, plants, etc.), and combinatorial libraries comprised of random peptides, oligonucleotides, or small organic molecules. In a particular embodiment, the assays herein are used to screen antibody libraries including, without limitation, naive human, recombinant, synthetic, and semi-synthetic antibody libraries. The antibody library can, for example, be a phage display library, including monovalent libraries, displaying on average one single-chain antibody or antibody fragment per phage particle, and multi-valent libraries, displaying, on average, two or more antibodies or antibody fragments per viral particle. However, the antibody libraries to be screened in accordance with the present invention are not limited to phage display libraries. Other display techniques include, for example, ribosome or mRNA display (Mattheakis et al., Proc. Natl. Acad. Sci. U.S.A. 91:9022-9026, 1994; Hanes et al., Proc. Natl. Acad. Sci. U.S.A. 94:4937-4942, 1997), microbial cell display, such as bacterial display (Georgiou et al., Nature Biotech. 15:29-34, 1997), or yeast cell display (Kieke et al., Protein Eng. 10:1303-1310, 1997), display on mammalian cells, spore display, viral display, such as retroviral display (Urban et al., Nucleic Acids Res. 33:e35, 2005), display based on protein-DNA linkage (Odegrip et al., Proc. Acad. Natl. Sci. U.S.A. 101:2806-2810, 2004; Reiersen et al., Nucleic Acids Res. 33:e10, 2005), and microbead display (Sepp et al., FEBS Lett. 532:455-458, 2002).

The results obtained in the assays described above can be confirmed in in vitro and/or in vivo assays of neuron and/or axon degeneration. Alternatively, in vitro and/or in vivo assays of neuron and/or axon degeneration may be used as primary assays to identify inhibitors and antagonists as described herein.

i) Cell-Based and In Vitro Assays of Neuron or Axon Degeneration

After an agent is confirmed as reducing or inhibiting DLK phosphorylation, the inhibitors can be tested in models of neuron or axon degeneration, as described herein, as well as in appropriate animal model systems.

Exemplary assays for identifying and characterizing additional agents which inhibit or reduce DLK phosphorylation and thus inhibit neuron or axon degeneration, and which can be used in the methods of the invention, are described briefly as follows.

Assays for confirming that an agent which reduces or inhibits phosphorylation of DLK also inhibits neuron or axon degeneration, as well as for identifying additional agents for use in the methods of the present invention, are described in detail in the Examples below and are briefly summarized as follows. These assays include (i) anti-Nerve Growth Factor (anti-NGF) antibody assays, (ii) serum deprivation/potassium chloride (KCl) reduction assays, (iii) rotenone degeneration assays, (iv) retina optic nerve crush and (iv) vincristine degeneration assays. Additional assays for assessing neuron or axon degeneration that are known in the art can also be used in the invention.

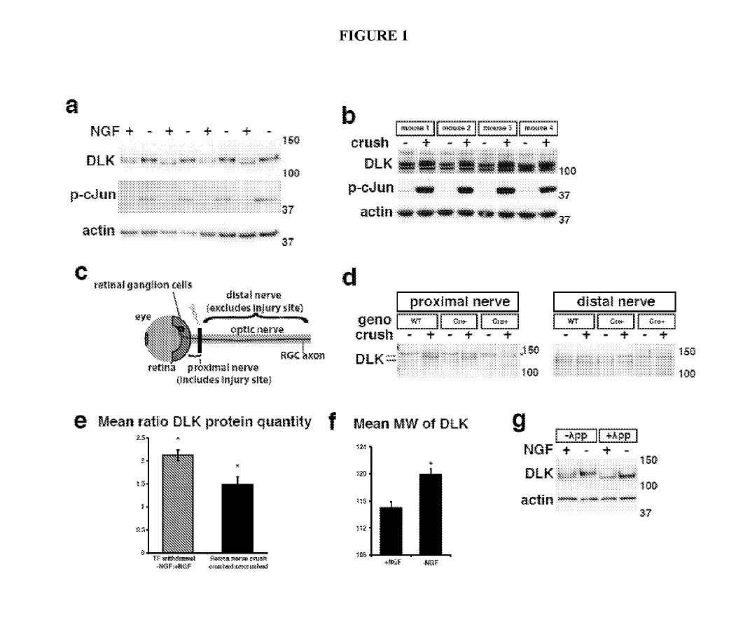

NGF is a small, secreted protein involved in differentiation and survival of target neurons. Treatment of cultured neurons with NGF results in proliferation of axons, while treating such neurons with anti-NGF antibodies results in axon degeneration. Treatment of neurons with anti-NGF antibodies also leads to several different morphological changes that are detectable by microscopy, and which can be monitored to observe the effects of candidate inhibitors. These changes include varicosity formation, loss of elongated mitochondria, accumulation of mitochondria in varicosities, cytoskeletal disassembly, and axon fragmentation. Agents that are found to counter any of the morphological changes induced by anti-NGF antibodies can be considered as candidate inhibitors of neuron or axon degeneration, which may, if desired, be tested in additional systems, such as those described herein. Additionally, as described in Example 1 and illustrated in, for example, FIGS. 1-3, NGF withdrawal leads to an increase in DLK protein. Thus, agents that are found to prevent an increase in DLK protein can be considered as a candidate agent for use in the methods of the present invention.

The serum deprivation/KCl reduction assay is based on the use of cultures of cerebellar granule neurons (CGN) isolated from mouse (e.g., P7 mouse) brains. In this assay, the neurons are cultured in a medium including KCl and then are switched to medium containing less KCl (Basal Medium Eagles including 5 mM KCl), which induces neuron degeneration. Agents that are found to block or reduce neuron degeneration upon KCl withdrawal, which can be detected by, for example, analysis of images of fixed neurons stained with a neuronal marker (e.g., anti-class III beta-tubulin) can be considered as candidate inhibitors of neuron or axon degeneration, which may, if desired, be tested in additional systems, such as those described herein.

Another model of neuron or axon degeneration involves contact of cultured neurons with rotenone (2R,6aS,12aS)-1,2,6,6a,12,12a-hexahydro-2-isopropenyl-8,9-dimethoxychrome-no[3,4-b]furo(2,3-h)chromen-6-one), which is a pesticide and insecticide that naturally occurs in the roots and stems of several plants, interferes with mitochondrial electron transport, and causes Parkinson's disease-like symptoms when injected into rats. Agents that are found to block or reduce degeneration of neurons cultured in the presence of rotenone, which can be detected by, for example, analysis of images of fixed neurons stained with, e.g., an antibody against neuron specific beta III tubulin, can be considered as candidate inhibitors of neuron or axon degeneration, which may, if desired, be tested in additional systems, such as those described herein.

An additional model of neuron or axon degeneration involves contact of cultured neurons with vincristine, an alkaloid that binds to tubulin dimers and prevents assemble of microtubule structures. Agents that are found to block or reduce degeneration of neurons cultured in the presence of vincristine, which can be detected by, for example, analysis of images of fixed neurons stained with, e.g., an antibody against neuron specific beta III tubulin, can be considered as candidate inhibitors of neuron or axon degeneration, which may, if desired, be tested in additional systems, such as those described herein.

The retinal optic nerve crush model of neuron or axon degeneration is described further in the Examples, but is also described in Quigley, H. A. et al. Retinal ganglion cell death in experimental glaucoma and after axotomy occurs by apoptosis. Investigative ophthalmology & visual science 36, 774-786 (1995) and Ghosh, A. S. et al. DLK induces developmental neuronal degeneration via selective regulation of proapoptotic JNK activity. The Journal of cell biology 194, 751-764 (2011), which are both incorporated herein by reference.

ii) Animal Models of Neuron or Axon Degeneration

In vivo assays for use in the invention include animal models of various neurodegenerative diseases, such as animal models of amyotrophic lateral sclerosis (ALS), Alzheimer's disease, Parkinson's disease, and multiple sclerosis (e.g., experimental autoimmune encephalitis (EAE) in mice). In addition, spinal cord and traumatic brain injury models can be used. Non-limiting examples of in vivo assays that can be used in characterizing agents for use in the invention are described as follows.

In the case of amyotrophic lateral sclerosis (ALS), a transgenic mouse that expresses a mutant form of superoxide dismutase 1 (SOD1) recapitulates the phenotype and pathology of ALS (Rosen et al., Nature 362(6415):59-62, 1993). In addition to the SOD1 mouse, several mouse models of amyotrophic lateral sclerosis (ALS) have been developed and can be used in the invention. These include motor neuron degeneration (Mnd), progressive motor neuropathy (pmn), wobbler (Bird et al., Acta Neuropathologica 19(1):39-50, 1971), and TDP-43 mutant transgenic mice (Wegorzewska et al., Proc. Natl. Acad. Sci. U.S.A., e-published on Oct. 15, 2009). In addition, a canine model has been developed and can be used in the invention (hereditary canine spinal muscular atrophy (HCSMA)).

Animal models that simulate the pathogenic, histological, biochemical, and clinical features of Parkinson's disease, which can be used in characterizing inhibitors for use in the methods of the present invention, include the reserpine (rabbit; Carlsson et al., Nature 180:1200, 1957); methamphetamine (rodent and non-human primates; Seiden et al., Drug Alcohol Depend 1:215-219, 1975); 6-OHDA (rat; Perese et al., Brain Res. 494:285-293, 1989); MPTP (mouse and non-human primates; Langston et al., Ann. Neurol. 46:598-605, 1999); paraquat/maneb (mouse; Brooks et al., Brain Res. 823:1-10, 1999 and Takahashi et al., Res. Commun. Chem. Pathol. Pharmacol. 66:167-170, 1989); rotenone (rat; Betarbet et al., Nat. Neurosci. 3:1301-1306, 2000); 3-nitrotyrosine (mouse; Mihm et al., J. Neurosci. 21:RC149, 2001); and mutated a-synuclein (mouse and Drosophila; Polymeropoulos et al., Science 276:2045-2047, 1997) models.

Genetically-modified animals, including mice, flies, fish, and worms, have been used to study the pathogenic mechanisms behind Alzheimer's disease. For example, mice transgenic for β-amyloid develop memory impairment consistent with Alzheimer's disease (Gotz et al., Mol. Psychiatry. 9:664-683, 2004). Models such as these may be used in characterizing the agents for use in characterizing agents for use in the present invention.

Several animal models are used in the art to study stroke, including mice, rats, gerbils, rabbits, cats, dogs, sheep, pigs, and monkeys. Most focal cerebral ischemia models involve occlusion of one major cerebral blood vessel such as the middle cerebral artery (see, e.g., Garcia, Stroke 15:5-14, 1984 and Bose et al., Brain Res. 311:385-391, 1984). Any of these models may also be used in the invention.

B. Making Antibody Agents

Antibodies which prevent or decrease phosphorylation of DLK; antibodies which bind certain phospho-forms of DLK; and antibodies identified by the binding and activity assays of the present invention can be produced by methods known in the art, including techniques of recombinant DNA technology.

1. Antigen Preparation

Soluble antigens or fragments thereof, optionally conjugated to other molecules, can be used as immunogens for generating antibodies. Exemplary sequences are described in the Examples, and can be used in the preparation of antigens for making antibodies for use in the invention. Other antigens and forms thereof useful for preparing antibodies will be apparent to those in the art.

2. Polyclonal Antibodies

Polyclonal antibodies are typically raised in animals by multiple subcutaneous (sc) or intraperitoneal (ip) injections of the relevant antigen and an adjuvant. It may be useful to conjugate the relevant antigen to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOCl₂, or R₁NCNR, where R and R₁ are different alkyl groups.

Animals are immunized against the antigen, immunogenic conjugates, or derivatives by combining, e.g., 100 μg or 5 μg of the protein or conjugate (for rabbits or mice, respectively) with 3 volumes of Freund's complete adjuvant and injecting the solution intradermally at multiple sites. One month later the animals are boosted with ⅕ to 1/10 of the original amount of peptide or conjugate in Freund's complete adjuvant by subcutaneous injection at multiple sites. Seven to 14 days later the animals are bled and serum is assayed for antibody titer. Animals are boosted until the titer plateaus. The animal can be boosted with a conjugate of the same antigen, but conjugated to a different protein and/or through a different cross-linking reagent. Conjugates also can be made in recombinant cell culture as protein fusions. Also, aggregating agents such as alum are suitably used to enhance the immune response.

3. Monoclonal Antibodies

Monoclonal antibodies may be made using the hybridoma method first described by Kohler et al., Nature 256:495, 1975, or may be made by recombinant DNA methods (see, e.g., U.S. Pat. No. 4,816,567). In the hybridoma method, a mouse or other appropriate host animal, such as a hamster or macaque monkey, is immunized as hereinabove described to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103, Academic Press, 1986).

The hybridoma cells thus prepared are seeded and grown in a suitable culture medium that can contain one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Exemplary myeloma cells are those that fuse efficiently, support stable high-level production of antibody by the selected antibody-producing cells, and are sensitive to a medium such as HAT medium. Among these, particular myeloma cell lines that may be considered for use are murine myeloma lines, such as those derived from MOPC-21 and MPC-11 mouse tumors available from the Salk Institute Cell Distribution Center, San Diego, Calif., USA, and SP-2 or X63-Ag8-653 cells available from the American Type Culture Collection, Manassas, Va., USA. Human myeloma and mouse-human heteromyeloma cell lines also have been described for the production of human monoclonal antibodies (Kozbor, J. Immunol. 133:3001, 1984; Brodeur et al., Monoclonal Antibody Production Techniques and Applications, pp. 51-63, Marcel Dekker, Inc., New York, 1987).

Culture medium in which hybridoma cells are growing is assayed for production of monoclonal antibodies directed against the antigen. The binding specificity of monoclonal antibodies produced by hybridoma cells can be determined by immunoprecipitation or by an in vitro binding assay, such as radioimmunoassay (RIA) or enzyme-linked immunoabsorbent assay (ELISA).

After hybridoma cells are identified that produce antibodies of the desired specificity, affinity, and/or activity, clones may be subcloned by limiting dilution procedures and grown by standard methods (Goding, Monoclonal Antibodies: Principles and Practice, pp. 59-103, Academic Press, 1986). Suitable culture media for this purpose include, for example, D-MEM or RPMI-1640 medium. In addition, the hybridoma cells may be grown in vivo as ascites tumors in an animal.

The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

DNA encoding the monoclonal antibodies is readily isolated and sequenced using conventional procedures (e.g., by using oligonucleotide probes that are capable of binding specifically to genes encoding the heavy and light chains of the monoclonal antibodies). The hybridoma cells serve as a source of such DNA. Once isolated, the DNA may be placed into expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. Recombinant production of antibodies and isolation of antibodies from libraries are described in more detail below.

4. Antibody Fragments Monoclonal Antibodies

In certain embodiments, an antibody for use in the methods herein is an antibody fragment. Antibody fragments include, but are not limited to, Fab, Fab′, Fab′-SH, F(ab′)₂, Fv, and scFv fragments, and other fragments described below. For a review of certain antibody fragments, see Hudson et al. Nat. Med. 9:129-134 (2003). For a review of scFv fragments, see, e.g., Pluckthün, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, Mass.; see, e.g., U.S. Pat. No. 6,248,516 B1).

Antibody fragments can be made by various techniques, including but not limited to proteolytic digestion of an intact antibody as well as production by recombinant host cells (e.g. E. coli or phage), as described herein.

5. Chimeric and Humanized Antibodies

In certain embodiments, an antibody for use in the methods herein is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall′Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

6. Human Antibodies

In certain embodiments, an antibody for use in the methods herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HuMAB® technology; U.S. Pat. No. 7,041,870 describing K-M MOUSE® technology, and U.S. Patent Application PUBLICATION NO. US 2007/0061900, DESCRIBING VELOCIMOUSE® TECHNOLOGY). HUMAN VARIABLE regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3502 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

7. Library-Derived Antibodies

Antibodies for use in the methods of the invention may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al. in Methods in Molecular Biology 178:1-37 (O'Brien et al., ed., Human Press, Totowa, N.J., 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, N.J., 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004).

In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

Antibodies or antibody fragments isolated from human antibody libraries are considered human antibodies or human antibody fragments herein.

8. Multispecific Antibodies

In certain embodiments, an antibody for use in the methods herein is a multispecific antibody, e.g. a bispecific antibody. Multispecific antibodies are monoclonal antibodies that have binding specificities for at least two different sites. In certain embodiments, one of the binding specificities is for DLK, phosphorylated DLK or a specific phospho-form of DLK and the other is for any other antigen. In certain embodiments, bispecific antibodies may bind to two different epitopes of DLK. Bispecific antibodies can be prepared as full length antibodies or antibody fragments.

Techniques for making multispecific antibodies include, but are not limited to, recombinant co-expression of two immunoglobulin heavy chain-light chain pairs having different specificities (see Milstein and Cuello, Nature 305: 537 (1983)), WO 93/08829, and Traunecker et al., EMBO J. 10: 3655 (1991)), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992)); using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g. Gruber et al., J. Immunol., 152:5368 (1994)); and preparing trispecific antibodies as described, e.g., in Tutt et al. J. Immunol. 147: 60 (1991).

Engineered antibodies with three or more functional antigen binding sites, including “Octopus antibodies,” are also included herein (see, e.g. US 2006/0025576A1).

The antibody or fragment for use in the methods herein also includes a “Dual Acting FAb” or “DAF” comprising an antigen binding site that binds to DLK, phosphorylated DLK or a specific phosphor-form of DLK as well as another, different antigen (see, US 2008/0069820, for example

9. Recombinant Methods and Compositions

Antibodies may be produced using recombinant methods and compositions, e.g., as described in U.S. Pat. No. 4,816,567. Such nucleic acid may encode an amino acid sequence comprising the VL and/or an amino acid sequence comprising the VH of the antibody (e.g., the light and/or heavy chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N.J., 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR⁻ CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003).

C. Methods and Compositions for Diagnostics and Detection

In certain embodiments, any of the agents provided herein is useful for detecting the presence of phosphorylated DLK or a specific phospho-form of DLK in a biological sample. The term “detecting” as used herein encompasses quantitative or qualitative detection. In certain embodiments, a biological sample comprises a cell or tissue, such as a neuron or portion thereof.

In one embodiment, an agent for use in a method of diagnosis or detection is provided. In a further aspect, a method of detecting the presence of phosphorylated DLK and/or a specific phosphorylated form of DLK (e.g., DLK phosphorylated at an amino acid residue selected from the threonine at position 43 of the human or murine DLK sequence (SEQ ID NOs:1 and 2, respectively); the serine at position 500 of the human DLK sequence (SEQ ID NO:1) and the serine at position 533 of the murine DLK sequence (SEQ ID NO:2); and any combination thereof) in a biological sample is provided. In certain embodiments, the method comprises contacting the biological sample with an antibody wherein specifically recognizes a phosphorylated form of DLK as described herein under conditions permissive for binding of the antibody to the phosphorylated form of DLK, and detecting whether a complex is formed between the antibody and the phosphorylated form of DLK. Such method may be an in vitro or in vivo method. The antibody is used to select neurons which have stress dependent and/or pro-apoptotic DLK activity and/or detect stress-dependent and/or pro-apoptotic DLK activity.

In certain embodiments, labeled antibodies for use in the methods of the invention are provided. Labels include, but are not limited to, labels or moieties that are detected directly (such as fluorescent, chromophoric, electron-dense, chemiluminescent, and radioactive labels), as well as moieties, such as enzymes or ligands, that are detected indirectly, e.g., through an enzymatic reaction or molecular interaction. Exemplary labels include, but are not limited to, the radioisotopes ³²P, ¹⁴C, ¹²⁵I, ³H, and ¹³¹I, fluorophores such as rare earth chelates or fluorescein and its derivatives, rhodamine and its derivatives, dansyl, umbelliferone, luceriferases, e.g., firefly luciferase and bacterial luciferase (U.S. Pat. No. 4,737,456), luciferin, 2,3-dihydrophthalazinediones, horseradish peroxidase (HRP), alkaline phosphatase, β-galactosidase, glucoamylase, lysozyme, saccharide oxidases, e.g., glucose oxidase, galactose oxidase, and glucose-6-phosphate dehydrogenase, heterocyclic oxidases such as uricase and xanthine oxidase, coupled with an enzyme that employs hydrogen peroxide to oxidize a dye precursor such as HRP, lactoperoxidase, or microperoxidase, biotin/avidin, spin labels, bacteriophage labels, stable free radicals, and the like.

D. Therapeutic Methods and Compositions

Any of the agents provided herein may be used in therapeutic methods.

In certain embodiments, the invention provides an agent for use in a method of treating an individual having a neurodegenerative disease, condition or disorder comprising administering to the individual an effective amount of the agent. In one such embodiment, the method further comprises administering to the individual an effective amount of at least one additional therapeutic agent, e.g., as described below. In further embodiments, the invention provides an agent for use in inhibiting or preventing neuronal degeneration. In certain embodiments, the invention provides an agent for use in a method of inhibiting or preventing neuronal degeneration in an individual comprising administering to the individual an effective amount of an agent to inhibit or reduce phosphorylation of DLK and thereby decreasing DLK protein stability. An “individual” according to any of the above embodiments is preferably a human.

An agent for use in the methods of the invention (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

Agents for use in the methods of the invention would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibody need not be, but is optionally formulated with one or more agents currently used to prevent or treat the disorder in question. The effective amount of such other agents depends on the amount of antibody present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

When the agent target is located in the brain, certain embodiments of the invention provide for the agent to traverse the blood-brain barrier. Certain neurodegenerative diseases are associated with an increase in permeability of the blood-brain barrier, such that the agent (e.g., an antibody or antigen-binding fragment) to be readily introduced to the brain. When the blood-brain barrier remains intact, several art-known approaches exist for transporting molecules across it, including, but not limited to, physical methods, lipid-based methods, and receptor and channel-based methods.

Physical methods of transporting agents such as an antibody or antigen-binding fragment across the blood-brain barrier include, but are not limited to, circumventing the blood-brain barrier entirely, or by creating openings in the blood-brain barrier. Circumvention methods include, but are not limited to, direct injection into the brain (see, e.g., Papanastassiou et al., Gene Therapy 9:398-406, 2002), interstitial infusion/convection-enhanced delivery (see, e.g., Bobo et al., Proc. Natl. Acad. Sci. U.S.A. 91:2076-2080, 1994), and implanting a delivery device in the brain (see, e.g., Gill et al., Nature Med. 9:589-595, 2003; and Gliadel Wafers™, Guildford Pharmaceutical). Methods of creating openings in the barrier include, but are not limited to, ultrasound (see, e.g., U.S. Patent Publication No. 2002/0038086), osmotic pressure (e.g., by administration of hypertonic mannitol (Neuwelt, E. A., Implication of the Blood-Brain Barrier and its Manipulation, Volumes 1 and 2, Plenum Press, N.Y., 1989)), permeabilization by, e.g., bradykinin or permeabilizer A-7 (see, e.g., U.S. Pat. Nos. 5,112,596, 5,268,164, 5,506,206, and 5,686,416), and transfection of neurons that straddle the blood-brain barrier with vectors containing genes encoding the antibody or antigen-binding fragment (see, e.g., U.S. Patent Publication No. 2003/0083299).

Lipid-based methods of transporting agents such as an antibody or antigen-binding fragment across the blood-brain barrier include, but are not limited to, encapsulating the antibody or antigen-binding fragment in liposomes that are coupled to antibody binding fragments that bind to receptors on the vascular endothelium of the blood-brain barrier (see, e.g., U.S. Patent Application Publication No. 2002/0025313), and coating the antibody or antigen-binding fragment in low-density lipoprotein particles (see, e.g., U.S. Patent Application Publication No. 2004/0204354) or apolipoprotein E (see, e.g., U.S. Patent Application Publication No. 2004/0131692).

Receptor and channel-based methods of transporting the antibody or antigen-binding fragment across the blood-brain barrier include, but are not limited to, using glucocorticoid blockers to increase permeability of the blood-brain barrier (see, e.g., U.S. Patent Application Publication Nos. 2002/0065259, 2003/0162695, and 2005/0124533); activating potassium channels (see, e.g., U.S. Patent Application Publication No. 2005/0089473), inhibiting ABC drug transporters (see, e.g., U.S. Patent Application Publication No. 2003/0073713); coating antibodies with a transferrin and modulating activity of the one or more transferrin receptors (see, e.g., U.S. Patent Application Publication No. 2003/0129186), and cationizing the antibodies (see, e.g., U.S. Pat. No. 5,004,697).

For the prevention or treatment of disease, the appropriate dosage of an antibody of the invention (when used alone or in combination with one or more other additional therapeutic agents) will depend on the type of disease to be treated, the type of antibody, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments. Depending on the type and severity of the disease, about 1 μg/kg to 15 mg/kg (e.g. 0.1 mg/kg-10 mg/kg) of antibody can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. One exemplary dosage of the antibody would be in the range from about 0.05 mg/kg to about 10 mg/kg. Thus, one or more doses of about 0.5 mg/kg, 2.0 mg/kg, 4.0 mg/kg or 10 mg/kg (or any combination thereof) may be administered to the patient. Such doses may be administered intermittently, e.g. every week or every three weeks (e.g. such that the patient receives from about two to about twenty, or e.g. about six doses of the antibody). An initial higher loading dose, followed by one or more lower doses may be administered. An exemplary dosing regimen comprises administering. However, other dosage regimens may be useful. The progress of this therapy is easily monitored by conventional techniques and assays.

E. Articles of Manufacture

In another aspect of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is an antibody of the invention. The label or package insert indicates that the composition is used for treating the condition of choice. Moreover, the article of manufacture may comprise (a) a first container with a composition contained therein, wherein the composition comprises an antibody of the invention; and (b) a second container with a composition contained therein, wherein the composition comprises a further cytotoxic or otherwise therapeutic agent. The article of manufacture in this embodiment of the invention may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### III. Examples

The following are examples of methods and compositions of the invention. It is understood that various other embodiments may be practiced, given the general description provided above.

### Materials and Methods

The following materials and methods were used in the Examples as described below.

Mouse Models

DLK heterozygous and knockout mice, DLK conditional knockout mice, Phr1^{mag} mice, and JNK2 knockout mice were generated as described Ghosh, A. S. et al. (2011), Watkins, T. A. et al. (2013), Lewcock, J. W. et al. Neuron 56, 604-620 (2007), and Sabapathy, K. et al. Current biology: CB 9, 116-125 (1999). JNK3 knockout mice were generated in C57B1/6 ES cells by genOway (Lyon, France www.genoway.com) by homologous recombination with a targeting vector. The targeting vector contained homology arms of 3.8 kb and 6.5 kb and replaced most of exon 11 with a neomycin resistance cassette. The deleted region includes the T-P-Y tripeptide dual phosphorylation motif required for JNK activity. The neo cassette insertion creates a frameshift when exons 10 and 12 are spliced together, producing an early stop codon in exon 14. Neomycin-resistant ES cells clones were screened by PCR and Southern blot to validate homologous recombination of the cassette. Determination of JNK3 genotype was carried out by PCR with following primers:

(SEQ ID NO: 7)
Primer 1: 5′-CCAGTAACATTGTAGTCAAGTCT-3′
(SEQ ID NO: 8)
Primer 2: 5′-TGGTCTTCCGCTTGGTAT-3′
(SEQ ID NO: 9)
Primer 3: 5′-CGCCTTCTATCGCCTTCT-3′

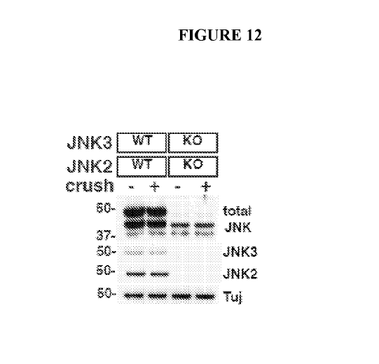

Primers 1 and 2 produce a 249-bp fragment in the WT allele and no product in the KO allele. Primers 1 and 3 produce a 435-bp fragment in the KO allele and no product in the WT allele. Blotting for JNK2 and JNK3 in retina samples from JNK2/3 double knockout and a littermate control shows loss of JNK2 and JNK3 protein in the knockout mice (FIG. 12).

USP9X conditional knockout mice were generated from C57BL/6 ES cells by Lexicon Pharmaceuticals. They contain a USP9X allele with loxp sites flanking exon 31, which encodes catalyic Cys 1560. Loxp sites were inserted by homologous recombination in ES cells using a FRT-flanked neomycin cassette with homology arms of 4.7 kb 5′ and 4.0 kb 3′ of exon 31. Neomycin-resistant ES cell clones were screened by Southern blot for homologous recombination of the cassette. Mice containing the floxed allele were crossed to a Flp deleter strain to remove the neomycin cassette. To achieve inducible recombination of the floxed USP9X allele, USP9X conditional knockout mice were crossed to a Rosa-Cre-ERT2 line, which contains an insertion of a construct encoding a Cre-estrogen receptor fusion protein into the rosa locus (Seibler, J. et al. Nucleic acids research 31, e12 (2003)). For DRG experiments, USP9X^{loxp/loxp}; Cre- and USP9X^{loxp/loxp}; Cre+ mice were crossed for timed pregnancies and embryos were genotyped for the presence or absence of Cre. Recombination in cultured DRGs was induced by adding 10 μM 4-hydroxytamoxifen (4-OHT, Sigma catalog # H7904) to the cells for 48 hours. 4-OHT was also added to Cre-control cells.

Primary Neuron Culture

Dorsal root ganglia were dissected from E12.5 to E13.5 mouse embryos, trypsinized (except in the case of explants), and cultured in F12 medium containing N3 supplement, 40 mM glucose, and 25 ng/mL NGF on chamber slides coated with poly-d-lysine and laminin (BioCoat, BD). The day after plating, 3 μM arabinofuranoside (AraC, Sigma) was added to the medium, removed two days later, and the medium was replaced with N3/F12/NGF without AraC. For NGF withdrawal experiments, after 4 to 5 days in vitro, medium was replaced with medium containing no NGF and 25 μg/mL anti-NGF antibody (Genentech) for between 1 and 3 hours.

For siRNA experiments, dissociated DRGs were transfected using the Amaxa nucleofection system (Lonza). JNK3 siRNA (sense 5′-ACA TCG TAG TCA AGT CTG ATT T-3′ (SEQ ID NO:10), antisense 5′-ATC AGA CTT GAC TAC GAT GTT T (SEQ ID NO:11)) was synthesized at Genentech (Kim, M. J. et al. Neuron 56, 488-502 (2007)). Control siRNA was ON-TARGETplus Non-targeting siRNA #1 from Dharmacon.

Western Blotting

DRG cultures were lysed by incubation on ice for 30 min. in buffer containing 50 mM Tris pH 7.5, 150 mM NaCl, 5 mM EDTA, and 0.1% Triton X-100. HEK 293T cells were lysed by incubation on ice for 30 min. in radioimmunoprecipitation assay (RIPA) buffer. Retina and nerve tissue samples were lysed in RIPA buffer using a TissueLyser (Qiagen) with a 3 mm tungsten carbide bead (Qiagen) for 6 min. Unless otherwise noted, all lysis solutions contained Complete protease inhibitor cocktail and PhosSTOP phosphatase inhibitor cocktail (Roche). Protein concentrations of 293T and retina lysates were determined by BCA assay (Pierce). Samples were loaded on NuPAGE 4-12% Bis-Tris gels (Invitrogen) and subjected to standard immunoblotting procedures. Except where noted, gels blotted for DLK were run in MOPS buffer (Invitrogen). Due to the large size of Phr1, samples blotted for Phr1 were run on 3-8% Tris-Acetate gels (Invitrogen). Blots were visualized with chemiluminescence and exposure to film. For relative protein expression and molecular weight quantifications, blots were also visualized on a Chemidoc (Bio-Rad). Quantifications were performed in ImageLab (Bio-Rad). Protein expression was standardized to a loading control (actin or tubulin). Molecular weight was standardized to Precision Plus Protein WesternC Standards (Bio-Rad).

Antibodies and Inhibitors

The following antibodies were used for staining and Western blotting: anti-DLK (1:1000, produced at Genentech according to reference Hirai, S. et al. Development 129, 4483-4495 (2002)); anti-p-JNK (1:250, Cell Signaling #9251); anti-p-cJun (1:250 for Western and 1:500 for staining, Cell Signaling #9261); anti-total JNK (1:500, Cell Signaling #9252); anti-JNK2 (1:500, Cell Signaling #4672); anti-JNK3 (1:500, Cell Signaling #2305); anti-f3-Tubulin (“Tuj”, 1:1000, Covance #MMS-435P-250); anti-actin (1:5000, BD #612656); anti-cleaved-caspase-3 (1:500, Cell Signaling #9664); Brn3 (1:100, Santa Cruz Biotechnology # sc-6026); γ-synuclein (1:200, Abcam # ab55424); NF-M (1:200, Covance MMS-583S). Anti-USP9X (rat monoclonal 4B3) was produced at Genentech and was raised against the 198 C-terminal amino acids of human USP9X. Antibodies to Phr1 were generated by immunizing rabbits with a fragment of Phr1 comprising amino acids D3812-Q3961, which consists of the DOC domain, expressed in baculovirus. Serum was then affinity purified using a column loaded with the same peptide prior to use. This portion of the protein is absent in Phr1^{mag} mutants. Antibodies to T43, S272, and S533 phosphorylation sites on DLK were generated through immunization of rabbits with the following peptides: PEKDL-pT-PTHVLQLHC (SEQ ID NO:12), HRDLK-pS-PNMLITYDC (SEQ ID NO: 14), RNVPQKL-pS-PHSKRPC (SEQ ID NO:13) and affinity purified prior to use.

The following inhibitors were used at the given concentrations: cycloheximide (5 μM, Calbiochem #239764); okadaic acid (“OA”, 200 nM, Sigma #08010), MG132 (30 μM, Fisher # NC9937881); JNK inhibitor V (“JNKV”, 10 μM, EMD #420129); JNK inhibitor VII (“JNKVII”, 10 μM, EMD #420134); JNK inhibitor VIII (“JNKVIII”, 10 μM, EMD #420135).

USP9X Activity Assay Using HA-Ub-Vinyl Sulfone

HA-tagged Ubiquitin Vinyl Sulfone was obtained from Enzo Life Sciences. The assay was performed as in Borodovsky, A. et al. Chemistry & biology 9, 1149-1159 (2002). Briefly, cultured DRGs were treated with anti-NGF or with NGF as controls. DRGs were then resuspended in lysis buffer (50 mM Tris pH 7.5, 5 mM MgCl₂, 250 mM sucrose, 1 mM DTT, 2 mM ATP, and 100 μM PMSF) and dounced for lysis. Lysates were then incubated with 6.6 μg/mL HA-Ubiquitin Vinyl Sulfone at 25° C. for 2 hours. N-ethyl-maleimide was added at 5 μM as a negative control. Reactions were stopped by boiling in sample buffer.

Real-Time Quantitative Reverse Transcription PCR (Real-Time qRT-PCR)

RNA samples from dissociated DRGs and retinas were collected using the RNeasy Plus Mini Kit (Qiagen). Pre-designed Taqman primer sets were ordered from Applied Biosystems. Catalog numbers for primer sets were as follows: DLK-Mm00437378_m1 (FAM labeled), GAPDH-4352339E (VIC labeled). Comparative Ct (ΔΔCt) assays were performed using the Taqman RNA-to-Ct One-Step Kit (Applied Biosystems #4392938) on a 7500 Real-Time PCR system and analyzed in 7500 Software. GAPDH endogenous control and DLK primers were multiplexed. All assays included five technical replicates. Error bars represent the standard deviation of the relative quantities calculated from these five technical replicates.

Lambda Protein Phosphatase Assay

Lambda protein phosphatase, 10X NEBuffer for PMP, and 10 mM MnCl₂ were all obtained from New England Biolabs. For DRGs, lysates were collected without phosphatase inhibitors or EDTA but otherwise under the same conditions as other DRG lysates in this manuscript. Lysates were incubated with 1X PMP buffer and 1 mM MnCl₂ with either 800 Units lambda protein phosphatase or the equivalent volume of 50% glycerol as a mock control at 30° C. for 30 min. Reactions were stopped by heating with sample buffer and loading on a gel.

Cycloheximide Timecourse to Determine DLK Stability

At time 0, DRG culture medium was replaced with medium containing no NGF, anti-NGF, and cycloheximide, as detailed in earlier Methods subheadings. Lysates were collected at the given timepoints and blotted for DLK. The experiment was performed three times and DLK was quantified relative to a loading control. The average quantity of DLK relative to the amount at time 0 was calculated for each timepoint. Linear regression and statistical analysis to compare the slopes of the two lines was performed in Graphpad Prism software.

Immunoprecipitations

For anti-ubiquitin immunoprecipitations (IPs), DRGs dissected from E12.5 CD-1 mice (Charles River Laboratories) were lysed as previously stated with the addition of 30 μM MG132 and 5 μM N-ethylmaleimide in the lysis buffer. Lysates were pre-cleared for 30 min. with Protein G conjugated Dynabeads (Life Technologies). 6 μg anti-ubiquitin antibody (clone FK2, Millipore) or equivalent.

In Vitro JNK Kinase Assay

Flag-tagged DLK^{S302A} was immunoprecipitated from 293T cell lysates using anti-Flag-conjugated magnetic beads (Sigma). Following washing, the DLK-bound Flag beads were incubated with 2,000 Units of lambda protein phosphatase, 1X PMP buffer, and 1X MnCl₂ for 30 min. at 30° C. to remove all phosphate groups from the purified DLK. The beads were then washed with buffer containing phosphatase inhibitors and split into two tubes with kinase reaction buffer (50 mM HEPES pH 7.2, 10 mM MgCl₂, 1 mM EGTA, 0.01% Triton-X-100, 2 mM DTT, 30 μM ATP). 126 ng GST-tagged human recombinant JNK3 (Millipore) was added to one of the two tubes and they were incubated at 30° C. for 90 min. DLK was eluted from the Flag beads by heating in sample buffer, and samples were loaded on a gel for blotting.

### Example 1

### Neuronal Stress Responses Lead to Increases in DLK Levels in Diverse Mammalian Stress Paradigms

Total DLK protein levels were examined to determine whether the total DLK level increases as a general response to neuronal stress, as has been observed in invertebrate (Xiong, X. et al. (2010)) and cell culture based models (Xu, Z. et al. (2001)). Two neuronal stress models were chosen which give reliable and reproducible neurodegeneration that is DLK dependent: the nerve growth factor (NGF) withdrawal assay of cultured embryonic sensory neurons, a model of developmental neuron cell death, and the retina optic nerve crush assay, a model for nerve injury and glaucoma^{3,18}. In cultured embryonic dorsal root ganglion cells (DRGs), DLK protein levels increased in response to NGF withdrawal by approximately 2-fold as compared to unstressed neurons cultured in the presence of NGF (FIG. 1a, e). Similarly, DLK levels in whole retinas increased within three days of optic nerve crush, by nearly 1.5-fold (FIG. 1b,e). In samples isolated from the optic nerve, the increase in DLK levels occurs only in the proximal side and not in the distal axons (FIG. 1c,d). In each case, DLK levels increased at a time point much earlier than the onset of neuronal degeneration, which begins after 16 hours in NGF withdrawal and after 3-7 days in retina nerve crush.

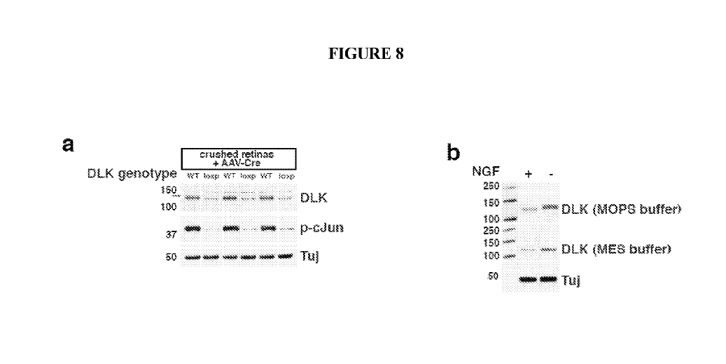

The increase in DLK protein quantity was accompanied by an increase in apparent molecular weight of DLK (FIG. 1a,b,d) of ˜5 kDa (FIG. 1f). Treatment of DRG lysates with lambda protein phosphatase to cleave phosphate groups equalized the molecular weights of DLK in +NGF and −NGF conditions (FIG. 1g), demonstrating that the mobility shift was the result of phosphorylation. In retina nerve crush, DLK is only phosphorylated in RGC's and not in other retinal cell types (FIG. 8a).

A DLK mobility shift has been observed in some instances (Xu, Z., Maroney, A. C., Dobrzanski, P., Kukekov, N. V. & Greene, L. A. Molecular and cellular biology 21, 4713-4724 (2001); Mata, M. et al. The Journal of biological chemistry 271, 16888-16896 (1996)), but not in others, and when it has been observed, the basis of this phenomenon has not been well understood. These conflicting results may be at least in part due to differences in SDS-PAGE buffer conditions used across research groups (FIG. 8b).

### Example 2

### DLK Protein is Stabilized in Response to Trophic Factor Withdrawal

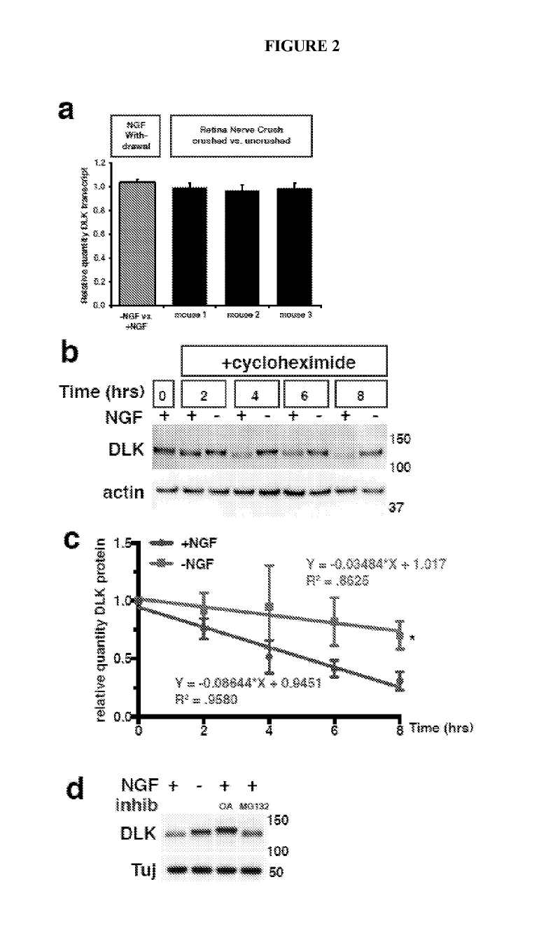

Possible mechanisms to explain the stress-induced DLK level rise include (1) increased transcription of Dlk, (2) increased protein translation, or (3) increased DLK stability. To address possibility 1, real-time qRT-PCR was performed to quantify the amount of DLK transcript in DRGs undergoing trophic factor withdrawal and in nerve crushed retinas. Neither condition showed a detectable change in DLK transcript levels compared to the unstressed controls (FIG. 2a), demonstrating that the rise in DLK protein levels is due to a post-transcriptional mechanism. To determine whether DLK stability is affected by neuronal stress, DRGs were treated with cycloheximide to inhibit protein translation in the presence or absence of NGF over a period of 8 hours and the amount of DLK remained over this time period was determined. A ˜2.5-fold increase in DLK stability was observed with NGF deprivation (FIG. 2b-c), indicating that the rise in DLK protein levels is a result of enhanced protein stability in response to neuronal stress.

In order to further investigate the role of phosphorylation in stress-dependent regulation of DLK, unstressed DRGs cultured in the presence of NGF were treated with okadaic acid, a broad phosphatase inhibitor, to enhance phosphorylation of DLK. This treatment was sufficient to increase both the apparent molecular weight and total levels of DLK, suggesting phosphorylation of DLK regulates protein stability (FIG. 2d). Treatment with MG132 produced an increase in DLK levels without an increase in DLK phosphorylation, suggesting that non-phosphorylated DLK is normally degraded by the proteasome (FIG. 2d).

### Example 3

### Neuronal Stress Regulates DLK Ubiquitination Via Modulation of Phr1

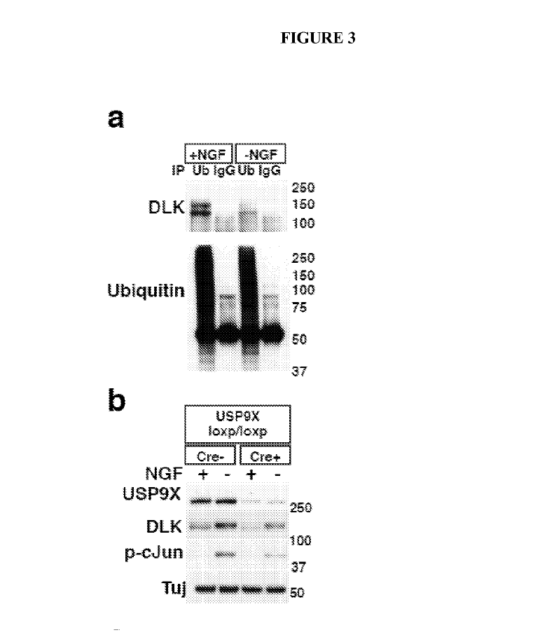

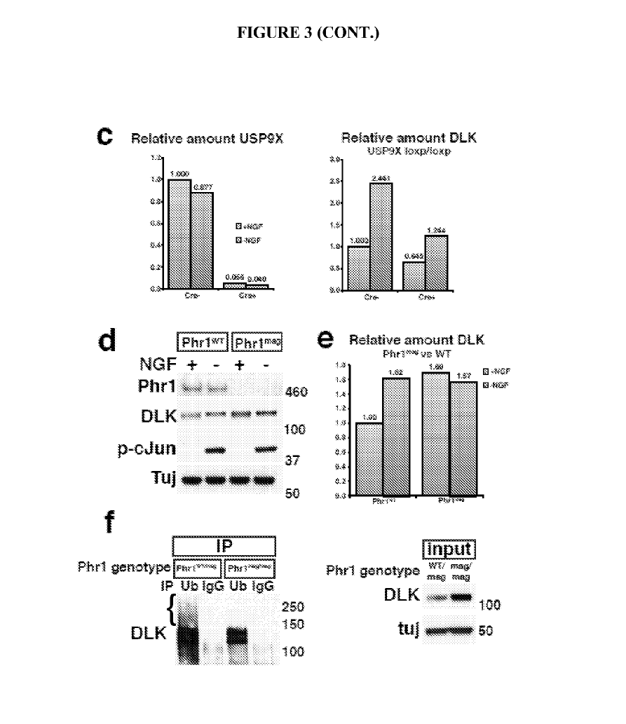

In order to determine whether neuronal stress decreases DLK ubiquitination to enhance DLK stability, ubiquitinated proteins were immunoprecipitated from +NGF and −NGF DRGs and it was found that DLK ubiquitination is markedly reduced in the −NGF condition (FIG. 3a). In invertebrate systems, the PHR family of E3 ubiquitin ligases (PAM/highwire/RPM-1) regulates DLK protein levels and a genetic interaction between the fat facets gene, which encodes a deubiquitinating enzyme (DUB), and the dlk homolog has been demonstrated (Collins, C. A., Wairkar, Y. P., Johnson, S. L. & DiAntonio, A. (2006); Nakata, K. et al. (2005); Xiong, X. et al. (2010)). However, because whole brain lysates from Phr1 KO mouse embryos show no change in DLK protein quantity (Bloom, A. J., Miller, B. R., Sanes, J. R. & DiAntonio, A. (2007)), it was unclear whether a similar pathway exists in vertebrates to regulate DLK. To directly investigate the role of the ubiquitin proteasome system in regulating DLK levels in mammalian neurons, DRGs were cultured that had loss of function alleles in the mouse homologs of these two genes.

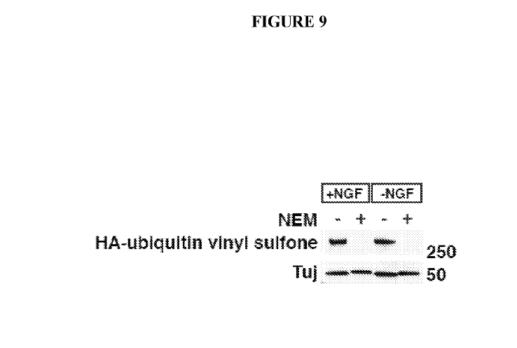

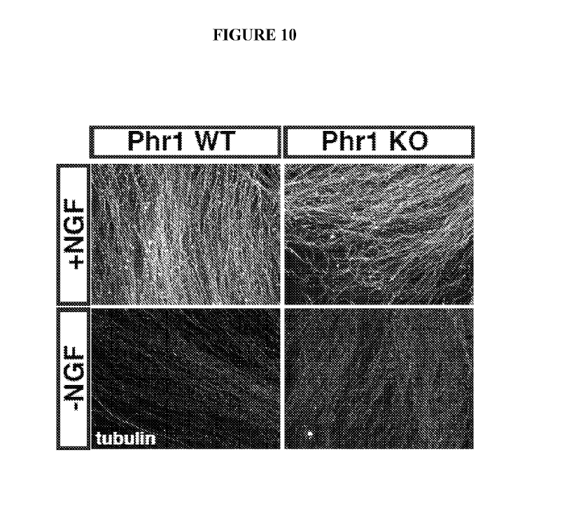

First DRGs from a conditional mouse knockout in USP9X, the closest mouse homolog to fat facets, were cultured. As would be predicted for knockout of a DUB that controls the turnover of DLK, a ˜35% reduction in DLK levels was observed in DRGs lacking USP9X (Cre+) versus Cre− DRGs (FIG. 3b,c). However, while the knockout of USP9X had an effect on the basal levels of DLK, the −NGF:+NGF ratio of DLK protein quantity in Cre− neurons was nearly identical to that in Cre+ neurons (FIG. 3c). Consistent with this observation, cross-linking with ubiquitin vinyl sulfone revealed that USP9X activity is unaltered by NGF withdrawal (FIG. 9). Therefore, we conclude that USP9X de-ubiquitinates DLK but is not required for the stress dependent change in DLK levels.

In contrast, DRGs homozygous for the loss-of-function Phr1^{mag} allele (Lewcock, J. W. et al. (2007)) displayed an increase in DLK levels in the presence of NGF compared to wild type controls while not affecting DLK levels following NGF deprivation, resulting in roughly equivalent DLK levels in stressed and non-stressed conditions (FIG. 3d,e). In addition, Phr1^{mag} homozygous neurons have lower quantities of ubiquitinated DLK (FIG. 3f). These observations, together with the observed decrease in DLK ubiquitination with neuronal stress (FIG. 3a) suggest that the change in ubiquitination of DLK is at least in part due to modulation of Phr1 activity or its interaction with DLK. However, the increase in amount of DLK in Phr1^{mag} neurons was not sufficient to drive downstream signaling and phosphorylation of downstream targets such as c-Jun. Thus, elevated DLK alone is not sufficient to induce downstream signaling events and additional inputs are required for DLK activation.

### Example 4

### DLK Activity and JNK Activity are Required for Stabilization of DLK

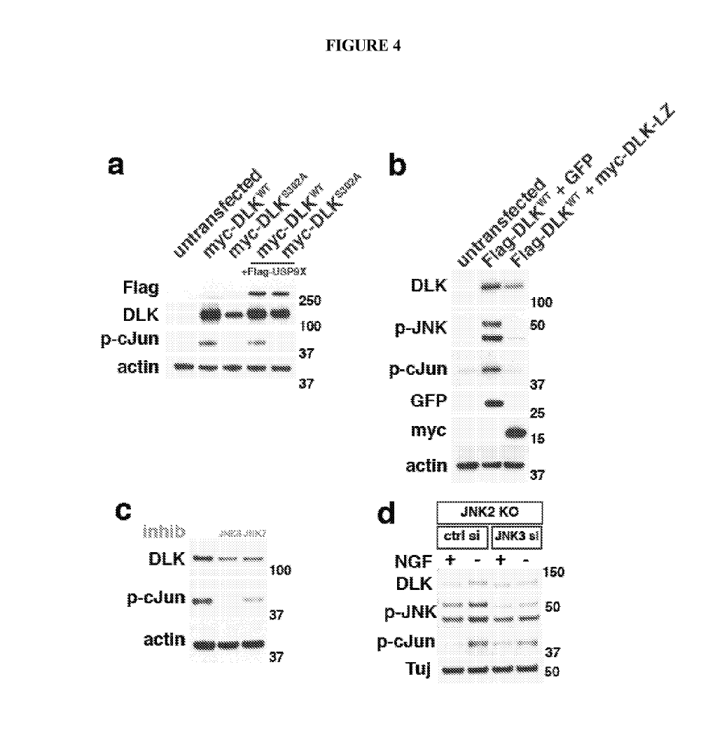

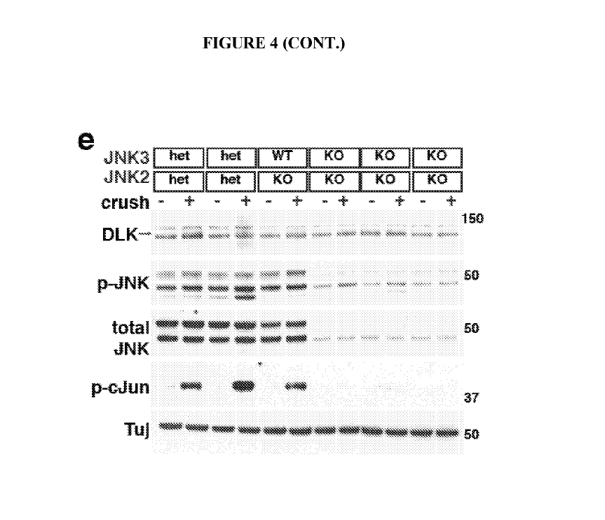

Neuronal stress leads to DLK phosphorylation and stabilization via decreased ubiquitination, and okadaic acid treatment also causes DLK phosphorylation and stabilization. This suggests a possible link between DLK phosphorylation and DLK stabilization. In order to determine how phosphorylation might regulate DLK protein stability, expression of Flag-tagged murine DLK in HEK 293T cells by transient transfection was examined. Wildtype DLK expressed in 293T cells is constitutively active because it dimerizes and auto-phosphorylates (Mata, M. et al. (1996)). In order to mimic, to some extent, the unstressed vs. stressed conditions in neurons, we made a kinase dead version of DLK by mutating phosphorylation sites in the putative activation loop that we identified by homology with MLK3 (Leung, I. W. & Lassam, N. The Journal of biological chemistry 276, 1961-1967(2001)). One such point mutant, DLK^{S302A}, was unable to cause phosphorylation of c-Jun when expressed, confirming that it lacks kinase activity. Interestingly, DLK^{S302A} expressed at lower levels than wild type DLK in HEK293T cells. Co-expression with USP9X increased expression of DLK^{S302A} to wild type levels, suggesting that the lower protein levels observed are due to increased ubiquitination of the inactive DLK (FIG. 4a). These data are consistent with our observations in neurons. To confirm that the reduction in protein expression observed with DLK^{S302A} was a result of a loss in kinase activity rather than an effect of this specific mutation, wildtype DLK with a truncated construct containing only the DLK leucine zipper, which acts as a dominant negative by preventing full length DLK dimerization (Nihalani, D., Merritt, S. & Holzman, L. B. The Journal of biological chemistry 275, 7273-7279 (2000)) were co-transfected. Similar to what was observed with DLK^{S302A}, reduction in DLK activity resulted in lowered expression of DLK when compared to DLK co-expressed with GFP (FIG. 4b).

As DLK pathway activity appeared necessary for protein stabilization, whether downstream signaling plays a role in DLK stability was examined. A stable cell line that expressed DLK in a doxicyclin-inducible fashion was used and cells were treated with two structurally distinct JNK inhibitors (FIG. 4c). Both JNK inhibitors were able to reduce levels of DLK protein.

To determine the relevance of this finding in a neuronal system, siRNA was used to knock down JNK3 expression in JNK2 knockout DRGs, removing the two JNK family members that regulate the majority of stress induced neuronal degeneration (Coffey, E. T. et al. The Journal of neuroscience: the official journal of the Society for Neuroscience 22, 4335-4345 (2002); Chang, L., Jones, Y., Ellisman, M. H., Goldstein, L. S. & Karin, M. Developmental cell 4, 521-533 (2003)). Compared to control siRNA, JNK3 knockdown attenuated the increase in DLK, though some change in DLK apparent molecular weight was still observed (FIG. 4d). It was hypothesized that JNK activity generates a feedback mechanism resulting in phosphorylation of specific sites on DLK that are required for DLK stabilization, though other JNK independent phosphorylation events also occur. In the retina nerve crush model, JNK2/3 double knockouts show no increase in DLK levels or molecular weight compared to littermate controls at 18 hours post-crush (FIG. 4e, arrow), demonstrating that JNK-dependent phosphorylation of DLK also occurs in an adult in vivo injury paradigm.

### Example 5

### Identification of Phosphorylation Sites Required for DLK Stabilization

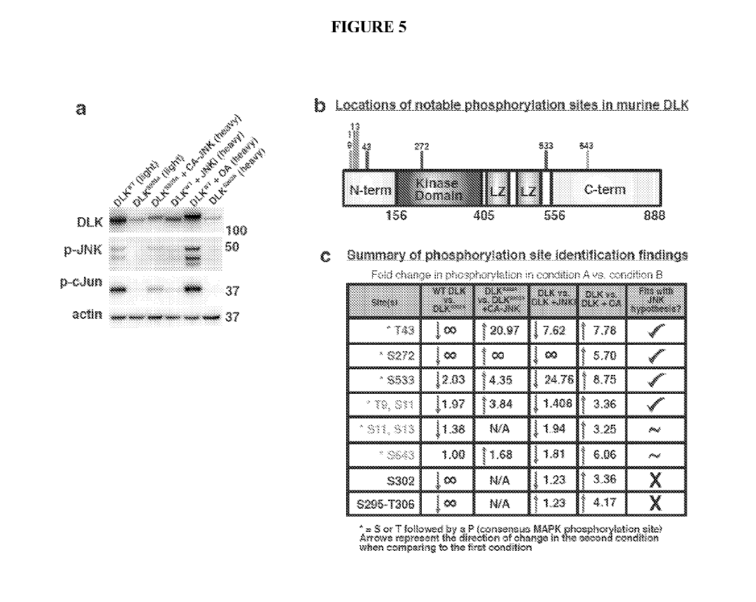

To identify functionally relevant phosphorylation sites on DLK, mass spectrometry in conjunction with stable isotope labeling by amino acids in cell culture (SILAC) was used. For these studies, 293T cells expressing FLAG-tagged DLK were cultured in SILAC media containing isotopically enriched (Heavy) versions of lysine (¹³C₆¹⁵N₂) and arginine (¹³C₆¹⁵N₄) or their unlabeled counterparts (Light). Four paired conditions (Light vs. Heavy) were established to dissect the effects of JNK and DLK activities on the overall abundance of DLK and the extent of DLK phosphorylation: 1) Wild-type (WT) DLK vs. DLK^{S302A}, 2) DLK^{S302A} vs. DLK^{S302A} co-expressed with a constitutively active JNK construct (Lei, K. et al. Molecular and cellular biology 22, 4929-4942 (2002)), 3) WT DLK vs. WT DLK with JNK inhibitor, and 4) WT DLK vs. WT DLK with okadaic acid (FIG. 5a).

A series of phosphopeptides on DLK were identified, the abundance of which changed in response to the conditions tested. Following correction for differences in overall abundance of DLK among conditions (see materials and methods), sites whose phosphorylation state changed in a manner consistent with DLK and JNK-dependent phosphorylation were identified (FIG. 5b,c). The top three sites in terms of magnitude of changes were T43 in the N-terminal domain, S272 in the kinase domain, and S533 immediately C-terminal to the leucine zipper domains. Changes were also observed for peptides containing multiple phosphorylation sites, in line with the predicted activities of JNK and DLK. Known phosphorylation sites within the kinase activation loop (S295-T306) were found to be dependent on the kinase activity of DLK but independent of JNK. This finding fits with a model in which JNK does not directly modulate the activity of DLK, but rather controls factors that affect DLK stability. Interestingly, within the sequence of DLK, the top identified sites contained a flanking proline consistent with a MAPK substrate motif (Songyang, Z. et al. Molecular and cellular biology 16, 6486-6493 (1996)).

### Example 6

### Identified Sites are Phosphorylated in Stabilized DLK

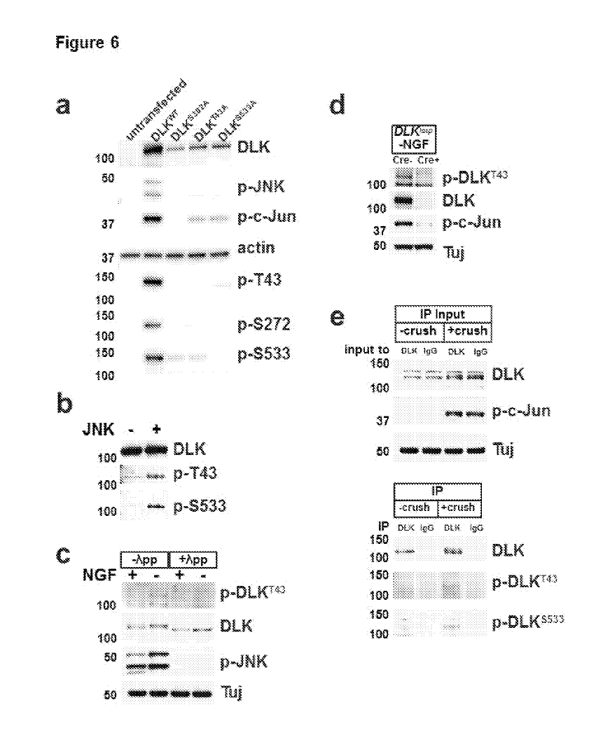

In order to determine the effect of phosphorylation of each of the top three identified sites on stability of DLK, alanine point mutants of each were expressed in 293T cells. 5272 was required for DLK activity as measured by c-Jun phosphorylation. It was not possible to distinguish changes in DLK stability resulting from the S272 mutant from those that occurred due to loss of kinase activity, thus this point mutation was not pursued further. Interestingly, T43 and S533 were not required for DLK activity, but DLK^{T43A} and DLK^{S533A} mutants expressed at lower levels than wild type DLK (FIG. 6a), consistent with a decrease in protein stability. Phospho-specific antibodies raised to T43, S272, and S533 residues demonstrated that not only is there a loss of phosphorylation of these sites in the point mutants, but also in the kinase dead DLK^{S302A}, consistent with the mass spectrometry results (FIG. 6a).

Whether phosphorylation of T43 and S533 occurs in a stress-dependent manner in neurons was examined. To answer this question, wild type DRGs were trophic factor deprived and blotted the lysates with antibodies specific to each of the two phosphorylation sites (FIG. 6b). The antibody targeting phospho-T43 showed immunoreactivity in the −NGF condition that was eliminated by lambda phosphatase treatment, demonstrating the specificity of this antibody for the phosphorylated target antigen. Blotting trophic factor deprived DLK loxp/loxp Cre− and Cre+ lysates demonstrated the specificity of this antibody for DLK (FIG. 6c). In addition, phosphorylation of both T43 and S533 can be detected in immunoprecipitated DLK from crushed retinas (FIG. 6d). Thus, T43 and S533 are phosphorylated following neuronal stress in vivo, consistent with the hypothesis that phosphorylation of these sites contributes to DLK stability in neurons. An in vitro kinase assay using purified JNK and DLK showed that both T43 and S533 can be phosphorylated directly by JNK (FIG. 6e). Thus, JNK may directly phosphorylate DLK in vivo.

### Example 7

### DLK Modulates Downstream Pro-Apoptotic Signaling in a Dose-Dependent Manner

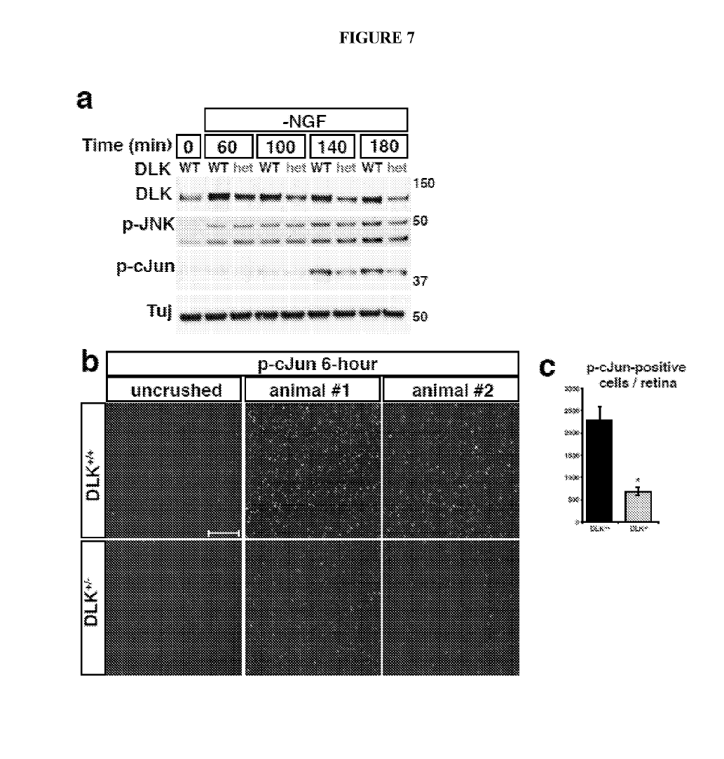

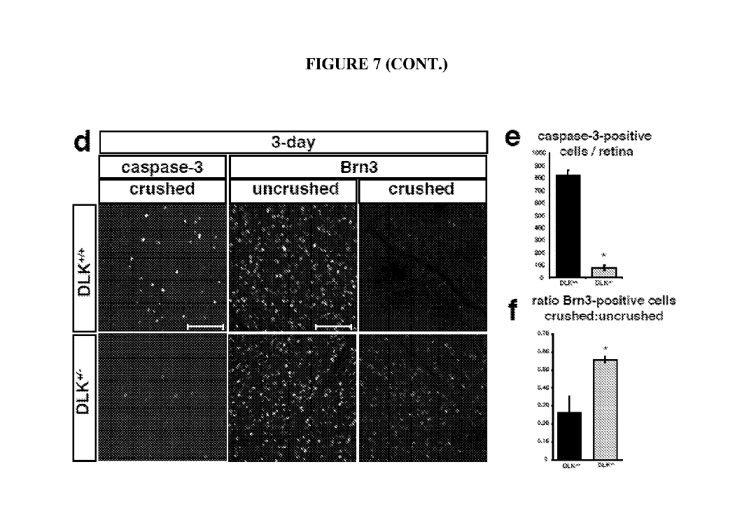

The observation that DLK protein quantity increases in response to trophic factor withdrawal and optic nerve crush prompted the examination of whether DLK protein levels directly affect the extent of downstream signaling induced by DLK following neuronal stress. To do this, we used DLK knockout heterozygotes that express roughly 50% the amount of DLK present in WT littermates. In a three-hour time course of NGF withdrawal (FIG. 7a), neurons heterozygous for the DLK KO allele showed lower levels of p-cJun and p-JNK compared to WT controls. Therefore, in DRGs the amount of DLK directly controls the amount of pro-apoptotic signaling.

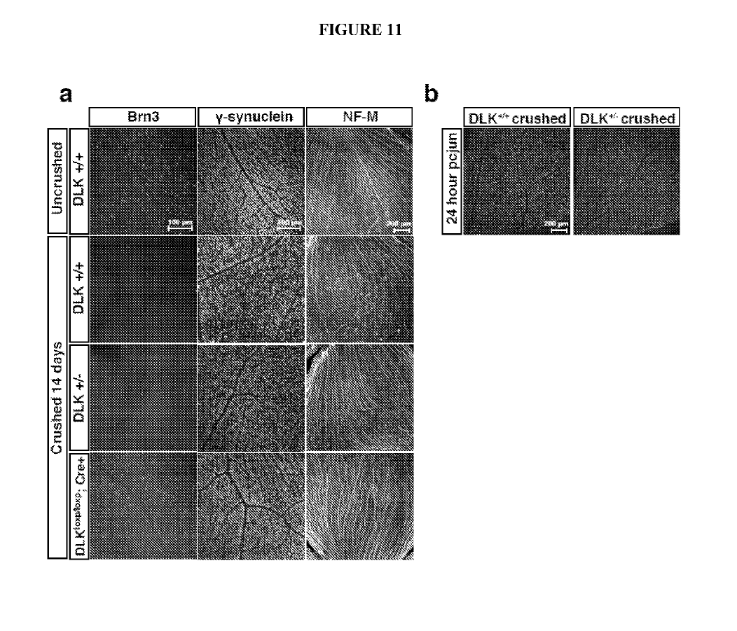

In retina nerve crush, there is a similar decrease in pro-apoptotic signaling in DLK heterozygous mice compared to DLK wild type mice. At 6 hours following optic nerve crush the number of p-cJun-positive nuclei in heterozygous crushed retinas is reduced by approximately 70% compared to the number found in WT retinas (FIG. 7b,c). Likewise, at three days post-crush, the amount of caspase-3-positive cells is reduced in DLK heterozygotes by >85% (FIG. 7d,e), while the total number of Brn3-positive nuclei is increased by >2-fold (FIG. 7d,f). Changes in these markers indicate that there is an abrogated stress response (Watkins, T. A. et al. In Press (2013)). At later timepoints, these markers become indistinguishable from what is observed in WT retinas, demonstrating that the reduced apoptotic signaling in the heterozygotes is a delay rather than an absolute reduction (FIG. 11). This differs from the DLK knockout, in which pro-apoptotic signaling following nerve crush is blocked (Watkins, T. A. et al. In Press (2013)). Thus, modulation of DLK levels tunes the amount and progression of downstream apoptotic signaling both in vivo and in vitro following neuronal stress.

It is proposed that DLK levels are tightly regulated under normal conditions via Phr1 and USP9X. In Phr1 mutants in particular, DLK levels increase in the absence of stress but this does not result in downstream signaling; therefore, additional factors are required for DLK activation. Neuronal stresses (e.g. NGF deprivation and injury) lead to activation of DLK kinase activity and phosphorylation of the downstream targets MKK4/7 and JNK. A JNK-dependent feedback mechanism then results in phosphorylation and stabilization of DLK. Stabilization occurs via a change in ubiquitination, as observed in NGF withdrawal. This change in ubiquitination likely occurs through a change in the activity of Phr1 or substrate availability of DLK for Phr1, although it is possible that additional E3 ubiquitin ligases also participate in ubiquitination of DLK. In any case, a decrease in ubiquitination due to positive feedback from JNK results in a rapid, switch-like, upregulation of DLK levels and activation of apoptosis and axon degeneration.

DLK kinase activity requires homodimerization and autophosphorylation (Nihalani, D., Merritt, S. & Holzman, L. B. (2000)), and autophosphorylation on the activation loop is required for kinase activity in the related kinase MLK3 (Leung, I. W. & Lassam, N. (2001)). Thus, it would be reasonable to assume that the phosphorylation of DLK observed in NGF withdrawal and nerve crush is the result of autophosphorylation on the DLK activation loop, or phosphorylation of the DLK activation loop by an upstream activating kinase. This is indeed likely to explain the observation that JNK inhibition does not completely inhibit phosphorylation of all sites within DLK following neuronal stress (FIG. 4). Mechanistic understanding of kinase regulation has largely focused on phosphorylation of the activation loops of numerous kinases. The above experiments provide evidence of a distinct mechanism in which DLK phosphorylation on specific residues outside the activation loop requires a downstream kinase. Phosphorylation of these residues affects stability of DLK without affecting DLK activity.

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1. A method for decreasing dual leucine zipper kinase (DLK) stability in a neuron comprising administering to a neuron, or portion thereof, an agent which decreases or inhibits the phosphorylation of DLK and decreases the stability of DLK.

2. A method for decreasing or inhibiting the phosphorylation of certain amino acid residues of dual leucine zipper kinase (DLK) comprising administering to a neuron or portion thereof an agent which decreases or inhibits the phosphorylation of DLK, wherein the decrease or inhibition of phosphorylation results in a decrease of DLK protein stability.

3. A method for inhibiting or preventing neuronal degeneration in a patient wherein the method comprises administering to a patient an agent which decreases or inhibits phosphorylation of dual leucine zipper kinase (DLK), wherein the decrease or inhibition of phosphorylation decreases the stability of DLK.

4. The method of any one of claims 1-3, wherein the agent decreases or inhibits the phosphorylation of a specific DLK amino acid residue.

5. The method of claim 4, wherein the specific DLK amino acid residue is selected from: threonine at position 43 (T43) of SEQ ID NO:1 (human); threonine at position 43 of SEQ ID NO:2 (mouse); serine at position 500 (S500) of SEQ ID NO:1 (human); serine at position 533 (S533) of SEQ ID NO:2 (mouse); and equivalent residues in DLK from other species or isoforms.

6. The method of any one of claims 1-3, wherein the agent is selected from an antibody, a small molecule, a polypeptide, and a short interfering RNA (siRNA).

7. The method of any one of claims 1-3, wherein the agent is an antibody.

8. The method of claim 7, wherein said antibody is selected from a polyclonal antibody, monoclonal antibody, chimeric antibody, humanized antibody, Fv fragment, Fab fragment, Fab′ fragment, and F(ab′)₂ fragment.

9. The method of any one of claims 1-3, wherein the neuron or portion thereof is present in a human subject, in a nerve graft or a nerve transplant, or is ex vivo or in vitro.

10. The method of any one of claims 1-3, wherein the neuron is selected from the group consisting of cerebellar granule neurons, dorsal root ganglion neurons, retinal ganglion cell neurons and cortical neurons.

11. The method of any one of claims 1-3, wherein the decrease or inhibition of DLK phosphorylation does not affect DLK kinase activity.

12. The method of any one of claims 1-3, wherein the agent is an inhibitor of JNK.

13. The method of any one of claims 1-3, wherein the method further comprises administering an inhibitor of JNK.

14. The method of claim 13, wherein the inhibitor of JNK inhibits a JNK selected from JNK1; JNK2; JNK3; and any combination of JNK1, JNK2 and JNK3.

15. The method of claim 13, wherein the inhibitor of JNK is selected from JNK Inhibitor V, JNK Inhibitor VII (TAT-TI-JIP₁₅₃₋₁₆₃), JNK Inhibitor VIII and siRNA.

16. The method of claim 3, wherein the patient is suffering from a disease or condition selected from Alzheimer's Disease, Parkinson's disease, Parkinson's-plus diseases, amyotrophic lateral sclerosis (ALS), trigeminal neuralgia, glossopharyngeal neuralgia, Bell's Palsy, myasthenia gravis, muscular dystrophy, progressive muscular atrophy, primary lateral sclerosis (PLS), pseudobulbar palsy, progressive bulbar palsy, spinal muscular atrophy, inherited muscular atrophy, invertebrate disk syndromes, cervical spondylosis, plexus disorders, thoracic outlet destruction syndromes, peripheral neuropathies, prophyria, Huntington's disease, multiple system atrophy, progressive supranuclear palsy, corticobasal degeneration, dementia with Lewy bodies, frontotemporal dementia, demyelinating diseases, Guillain-Barré syndrome, multiple sclerosis, Charcot-Marie-Tooth disease, prion disease, Creutzfeldt-Jakob disease, Gerstmann-Sträussler-Scheinker syndrome (GSS), fatal familial insomnia (FFI), bovine spongiform encephalopathy, Pick's disease, epilepsy, and AIDS demential complex, chronic pain, fibromyalgia, spinal pain, carpel tunnel syndrome, pain from cancer, arthritis, sciatica, headaches, pain from surgery, muscle spasms, back pain, visceral pain, pain from injury, dental pain, neuralgia, such as neuogenic or neuropathic pain, nerve inflammation or damage, shingles, herniated disc, torn ligament, and diabetes, peripheral neuropathy or neuralgia caused by diabetes, cancer, AIDS, hepatitis, kidney dysfunction, Colorado tick fever, diphtheria, HIV infection, leprosy, lyme disease, polyarteritis nodosa, rheumatoid arthritis, sarcoidosis, Sjogren syndrome, syphilis, systemic lupus erythematosus, or oramyloidosis, nerve damage caused by exposure to toxic compounds, heavy metals, industrial solvents, drugs, chemotherapeutic agents, dapsone, HIV medications, cholesterol lowering drugs, heart or blood pressure medications, or ormetronidazole, injury to the nervous system caused by physical, mechanical, or chemical trauma, schizophrenia, delusional disorder, schizoaffective disorder, schizopheniform, shared psychotic disorder, psychosis, paranoid personality disorder, schizoid personality disorder, borderline personality disorder, anti-social personality disorder, narcissistic personality disorder, obsessive-compulsive disorder, delirium, dementia, mood disorders, bipolar disorder, depression, stress disorder, panic disorder, agoraphobia, social phobia, post-traumatic stress disorder, anxiety disorder, and impulse control disorders, glaucoma, lattice dystrophy, retinitis pigmentosa, age-related macular degeneration (AMD), photoreceptor degeneration associated with wet or dry AMD, other retinal degeneration, optic nerve drusen, optic neuropathy, and optic neuritis.

17. A method for detecting stress dependent or pro-apopototic DLK activity in a neuron comprising: (a) contacting a biological sample with an antibody which specifically recognizes a phosphorylated form of DLK; and (b) detecting binding of the antibody to the phosphorylated form of DLK within the biological sample, wherein binding by the antibody indicates stress dependent or pro-apoptotic DLK activity.

18. The method of claim 17, further comprising measuring the binding of the antibody to the phosphorylated form of DLK, wherein an increase in binding of the antibody in the biological sample relative to a control is indicative of stress dependent or pro-apoptotic DLK activity.

19. The method of claim 17 or 18, wherein the biological sample comprises biological material selected from a neuron, neuronal cell lysate and DLK purified from a neuron.

20. The method of claim 17, wherein the antibody specifically binds to DLK phosphorylated at an amino acid residue selected from: threonine at position 43 (T43) of SEQ ID NO:1 (human); threonine at position 43 of SEQ ID NO:2 (mouse); serine at position 500 (S500) of SEQ ID NO:1 (human); serine at position 533 (S533) of SEQ ID NO:2 (mouse); and equivalent residues in DLK from other species or isoforms.

21. The method of claim 3, wherein the phosphorylation of DLK is in response to neuronal stress or injury.
